(19) United States
(12) Patent Application Publication
(10) Pub. No.: US 2018/0055465 A1
(43) Pub. Date: Mar. 1, 2018

(54) RADIOGRAPHIC IMAGE CAPTURING STAND

(71) Applicant: KONICA MINOLTA, INC., Tokyo (JP)

(72) Inventors: Shingo NAKAYAMA, Tokyo (JP); Takatyuki YOSHIMURA, Tokyo (JP); Kenichi YANAGISAWA, Tokyo (JP)

(73) Assignee: KONICA MINOLTA, INC., Tokyo (JP)

(21) Appl. No.: 15/683,102

(22) Filed: Aug. 22, 2017

(30) Foreign Application Priority Data
Aug. 23, 2016 (JP) 2016-162316

(51) Int. Cl. A61B 6/00

(52) U.S. Cl. CPC A61B 6/4405; A61B 6/4266

(57) ABSTRACT

A radiographic image capturing stand includes: a plurality of radiographic image capturing parts, each of which forms a radiographic image by being exposed to a radiation ray, and the radiographic image capturing stand being capable of switching between a single image capturing state in which image capturing is performed with the single radiographic image capturing part held with respect to one exposure to a radiation ray, and a long-length image capturing state in which image capturing is performed with the plurality of radiographic image capturing parts held side by side with respect to one exposure to a radiation ray.

Japanese Patent Application No. 2016-162316 filed on Aug. 23, 2016, including description, claims, drawings, and abstract the entire disclosure is incorporated herein by reference in its entirety.

### BACKGROUND

### Technological Field

The present invention relates to a radiographic image capturing stand, and in particular to a radiographic image capturing stand that is also capable of performing, what is called, one-shot long-length image capturing.

Description of the Related art

As an image capturing stand for performing relatively wide range radiographic image capturing (that is to say, so-called long-length image capturing), for example, the whole spinal column, or the whole lower limbs, of a subject that is an object, by using a radiographic image capturing device (Flat Panel Detector), there is under development an image capturing stand having a holder in which a plurality of radiographic image capturing devices are arranged side by side in a direction (up-and-down direction) of the body axis of a subject beforehand (refer to, for example, JP 2012-180239 A, etc.).

In addition, when long-length image capturing is performed by using the image capturing stand in this manner, positioning of the subject and each radiographic image capturing device in the holder is performed, and subsequently the plurality of radiographic image capturing devices are irradiated with a radiation ray only once (that is to say, one shot) through the subject from a radiation irradiation device, thereby enabling long-length image capturing to be performed.

It should be noted that irradiating a plurality of radiographic image capturing devices with a radiation ray only once through a subject to perform long-length image capturing in this manner is hereinafter referred to as “one-shot long-length image capturing”.

Moreover, there is also proposed another image capturing stand, wherein the image capturing stand has a holder that stores and holds a single radiographic image capturing device, the lower part of the outside front surface of the holder is provided with a hook for suspending another radiographic image capturing device, and only when long-length image capturing is required, the another radiographic image capturing device is suspended from the hook to enable one-shot long-length image capturing (refer to, for example, JP 2011-072775 A).

However, a holder of an image capturing stand disclosed in JP 2012-180239 A is vertically long, and thus the height of the upper end part thereof is high. Therefore, as shown in, for example, FIG. 32B, in the case of a holder H1 that stores a usual single radiographic image capturing device, a subject with short stature, such as a child, can be subjected to image capturing in a state in which the chin of the subject is placed on the upper end part of the holder H1 and the body of the subject nears the holder H1. However, as shown in FIGS. 32A and 33, in the case of a holder H2 that stores a plurality of radiographic image capturing devices, the following problem arises: the subject with short stature cannot be subjected to image capturing in a state in which the chin of the subject is placed on the upper end part of the holder H2 and the body of the subject nears the holder H2.

In addition, the holder of the image capturing stand disclosed in JP 2012-180239 A is vertically long. Accordingly, as shown in, for example, FIG. 34, in the case of the holder H1 that stores the usual single radiographic image capturing device, a free space can be easily maintained under the holder H1. Therefore, even in the case of a subject using a wheelchair, the body of the subject and the wheelchair can be easily placed under the holder H1, and the subject can be subjected to image capturing in a state in which the body of the subject nears the holder H1. However, in the case of the holder H2 that stores a plurality of radiographic image capturing devices, the following problem arises: as shown in FIG. 35, since a lower region is blocked by the holder H2, a free space cannot be maintained under the holder H2, and consequently the subject cannot be subjected to image capturing in a state in which the body of the subject nears the holder H2.

Moreover, the above-described problems require a radiographic image capturing room to be provided with an image capturing stand having a holder that stores a usual single radiographic image capturing device, and an image capturing stand having a long holder that stores a plurality of radiographic image capturing devices. This produces a problem of occupying a very large installation space.

Meanwhile, when the image capturing stand disclosed in JP 2011-072775 A is in a state in which no radiographic image capturing device is suspended from the hook, the image capturing stand functions in the same manner as that of a usual image capturing stand. Therefore, the image capturing stand is also capable of easily coping with image capturing of a subject with short stature and a subject using a wheelchair. However, when this image capturing stand performs one-shot long-length image capturing, the image capturing stand uses a radiographic image capturing device suspended from the hook, and therefore the posture of the image capturing stand gets unstable. When one-shot long-length image capturing is performed, an image captured by an internal radiographic image capturing device and an image captured by an external radiographic image capturing device are combined by image processing to form one image. Therefore, one-shot long-length image capturing requires that relative arrangement of the two radiographic image capturing devices, directions thereof and the like are stably fixed. However, there arises a problem that when one radiographic image capturing device is unstable, it is not possible to obtain an excellent captured image by synthesizing two captured images.

### SUMMARY

The present invention has been made taking the above-described problems into consideration, and an object of the present invention is to provide a radiographic image capturing stand.

To achieve the abovementioned object, according to an aspect of the present invention, a radiographic image capturing stand reflecting one aspect of the present invention comprises: a plurality of radiographic image capturing parts, each of which forms a radiographic image by being exposed to a radiation ray, and the radiographic image capturing stand being capable of switching between a single image capturing state in which image capturing is performed with the single radiographic image capturing part held with respect to one exposure to a radiation ray, and a long-length image capturing state in Which image capturing is performed with the plurality of radiographic image capturing parts held side by side with respect to one exposure to a radiation ray.

### BRIEF DESCRIPTION OF THE DRAWING

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a radiographic image capturing system according to a first embodiment;

FIG. 2 is a perspective view illustrating an external appearance of a radiographic image capturing device;

FIG. 3 is a block diagram illustrating an equivalent circuit of the radiographic image capturing device;

FIG. 4 is a diagram illustrating a configuration example of a sensor panel of the radiographic image capturing device;

FIG. 5A is a perspective view illustrating a radiographic image capturing stand in a long-length image capturing state;

FIG. 5B is a perspective view illustrating a radiographic image capturing stand in a single image capturing state;

FIG. 6A is a perspective view illustrating a coupler in a non-coupled state;

FIG. 6B is a perspective view illustrating the coupler in a coupled state;

FIG. 7A is a side view illustrating the radiographic image capturing stand from which a second holder is disconnected;

FIG. 7B is a side view illustrating the radiographic image capturing stand to which the second holder is connected;

FIG. 7C is a side view illustrating the radiographic image capturing stand to which the second holder is connected with the height adjusted;

FIG. 8 is a front view illustrating the radiographic image capturing stand that inclinably supports first and second holders;

FIG. 9A is a perspective view illustrating the radiographic image capturing stand from which the second holder is disconnected;

FIG. 9B is a perspective view illustrating h radiographic image capturing stand with the first holder kept in a horizontal state;

FIG. 9C is a perspective view illustrating the radiographic image capturing stand to which the second holder is further connected;

FIG. 10A is a perspective view illustrating the radiographic image capturing stand in a state in which a subject is holding on to a second holding bar;

FIG. 10B is a perspective view illustrating the radiographic image capturing stand in a state in which the subject is holding on to a first holding bar;

FIG. 11A is a perspective view illustrating the radiographic image capturing stand in a state in which the subject is holding on to the second holding bars at both right and left sides;

FIG. 11B is a perspective view illustrating the radiographic image capturing stand in a state in which one of the second holding bars is retracted downward, and the second holder is then moved;

FIG. 12 is a perspective view illustrating the second holding bar, a height adjusting mechanism, and a posture adjusting mechanism;

FIG. 13 is a perspective view illustrating the height adjusting mechanism;

FIG. 14 is a perspective view illustrating the posture adjusting mechanism;

FIG. 15A is a side view illustrating a non-engagement state in which two members are not engaged by a toggle-type locking lever used as an alternative to an engagement screw;

FIG. 15B is a side view illustrating an engagement state in which the two members are engaged;

FIG. 16A is a perspective view illustrating the front surface side of a moving auxiliary tool;

FIG. 16B is a perspective view illustrating the back surface side of the moving auxiliary tool;

FIG. 17A is a perspective view illustrating a state in which a movable handle of the moving auxiliary tool is turned down;

FIG. 17B is a perspective view illustrating a state in which the whole moving auxiliary tool is brought down;

FIG. 18A is a side view illustrating the moving auxiliary tool;

FIG. 18B is a side view illustrating a state in which an auxiliary caster and a support leg part are pulled out to incline the whole moving auxiliary tool;

FIG. 19 is a perspective view illustrating the moving auxiliary tool in a state in which the second holder and a holding frame are inclined;

FIG. 20A is a perspective view illustrating a single image capturing state of a radiographic image capturing stand according to a second embodiment;

FIG. 20B is a side view of the radiographic image capturing stand;

FIG. 21A is a perspective view illustrating a single image capturing state of a radiographic image capturing stand according to a third embodiment;

FIG. 21B is a perspective view illustrating a state in which a second holder is expanded right and left;

FIG. 22A is a perspective view illustrating a long-length image capturing state of the radiographic image capturing stand;

FIG. 22B is a perspective view illustrating a state in which a direction of only one second holder is changed;

FIG. 23A is a perspective view illustrating a state in which all holders of the radiographic image capturing stand faces upward;

FIG. 23B is a perspective view illustrating a state in which directions in which the second holders on the right and left sides face respectively differ from a direction in which the first holder faces;

FIG. 24A is a side view illustrating the radiographic image capturing stand at the time of single image capturing of a child or a subject with short stature;

FIG. 24B is a side view illustrating the radiographic image capturing stand at the time of single image capturing of a subject using a wheelchair;

FIG. 25 is a perspective view illustrating a long-length image capturing state of a radiographic image capturing stand according to a fourth embodiment;

FIG. 26 is a perspective view illustrating a state in which each holder of the radiographic image capturing stand is turned;

FIG. 27 is a perspective view illustrating a single image capturing state of the radiographic image capturing stand;

FIG. 28 is a front view illustrating a state in which the second holder of the radiographic image capturing stand is turned about a shaft extending along the front-and-back direction;

FIG. 29 is a perspective view illustrating a state in which the second holder of the radiographic image capturing stand faces upward;

FIG. 30 is a side view of the radiographic image capturing stand at the time of single image capturing of a child or a subject with short stature;

FIG. 31 is a side view of the radiographic image capturing stand at the time of single image capturing of a subject using a wheelchair;

FIG. 32A is a side view illustrating a problem that arises when single image capturing of a child or a subject with short stature is performed by using a conventional holder for long-length image capturing;

FIG. 32B is a side view illustrating a case where single image capturing of a child or a subject with short stature is performed by using a conventional holder for single image capturing;

FIG. 33 is a perspective view illustrating a problem that arises when single image capturing of a child or a subject with short stature is performed by using a conventional holder for long-length image capturing;

FIG. 34A is a side view illustrating a case where single image capturing of the chest of a subject using a wheelchair is performed by using a conventional holder for single image capturing;

FIG. 34B is a side view illustrating a case where single image capturing of the back surface is performed; and

FIG. 35 is a side view illustrating a problem that arises when single image capturing of a subject using a wheelchair is performed by using a conventional holder for long-length image capturing.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

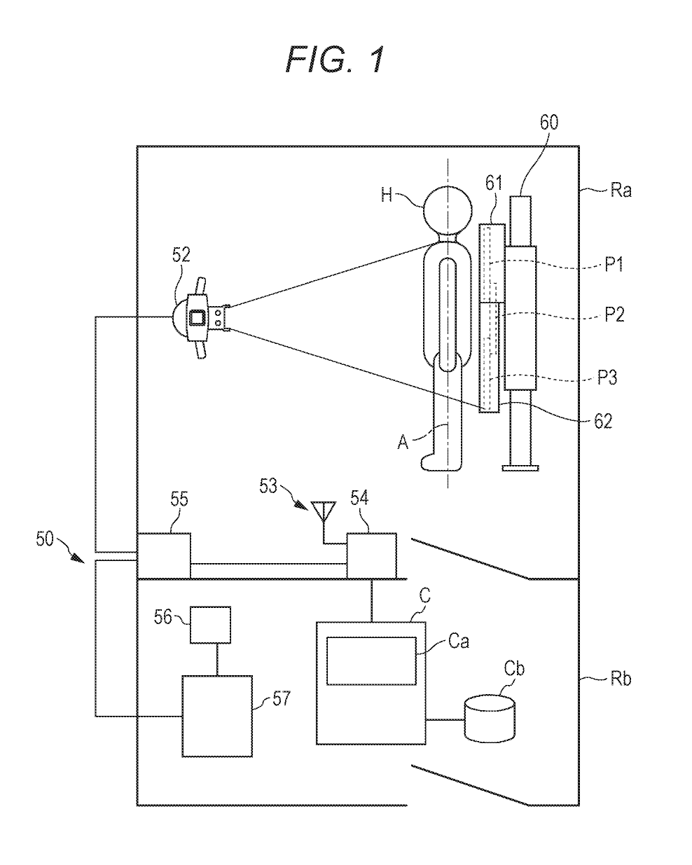

A radiographic image capturing system provided with a radiographic image capturing stand according to a first embodiment of the present invention will be described below with reference to the accompanying drawings. FIG. 1 is a diagram illustrating a configuration of a radiographic image capturing system 50 according to the present embodiment.

### Configuration and the Like of Radiographic Image Capturing System

As shown in FIG. 1, in the present embodiment, in order to switch between a tong-length image capturing state (that is to say, one-shot tong-length image capturing) and a single image capturing state (that is to say, general image capturing in which a single radiographic image capturing device is irradiated with a radiation ray only once to perform image capturing), a radiographic image capturing stand 60 that can be loaded with radiographic image capturing devices P1 to P3 as a plurality of radiographic image capturing parts is arranged in an image capturing room Ra. It should be noted that when the radiographic image capturing device is described without distinguishing among the radiographic image capturing devices P1 to P3, or when one radiographic image capturing device is represented, the radiographic image capturing device is hereinafter referred to as “radiographic image capturing device P”.

A radiation irradiation device 52 is provided in the image capturing room Ra. In addition, a relay 54 for relaying communications with each unit and the like inside the image capturing room Ra, and with each unit and the like outside the image capturing room Ra, is provided in the image capturing room Ra. Moreover, the relay 54 is provided with an access point 53 that enables the radiographic image capturing devices P1 to P3 to wirelessly transmit/receive image data D, a signal and the like. It should be noted that in FIG. 1, the radiographic image capturing system may be configured in such a manner that the relay 54 is connected by cable or the like to each of the radiographic image capturing devices P1 to P3, with which the image capturing stand 60 is loaded as described above, so as to enable wired communications The relay 54 is connected to a control unit 55 of the radiation irradiation device 52, and is connected to a console C.

As shown in FIG. 1, an operation desk 57 of the radiation irradiation device 52 is provided in a front room (also called an operation room or the like) Rb, and the operation desk 57 is provided with an exposure switch 56 that is operated by an operator such as a radiological technician to instruct the radiation irradiation device 52, for example, to start the irradiation with a radiation ray.

In addition, the console C that is composed of a computer and the like is provided in the front room Rb. In the computer, a central processing unit (CPU), a read only memory (ROM), a random access memory (RAM), an input-output interface and the like, which are not illustrated, are connected through buses.

The console C is provided with a display unit Ca that is formed of a cathode ray tube (CRT), a liquid crystal display (LCD) or the like. In addition, the console C is provided with an input unit such as a mouse and a keyboard (not illustrated). Moreover, a storage unit Cb composed of a hard disk drive (HDD) and the like is connected to, or built into, the console C. Further, although illustration is omitted, a hospital information system (HIS), a radiology information system (RIS), a picture archiving and communication system (PACS) and the like are connected to the console C through a network N or the like.

Incidentally, in the present embodiment, the console C is configured to function as an image processing device. Thus, when the console C functions as an image processing device, the console C will be described below as the image processing device. However, the image processing device may be configured separately from the console C.

### Radiographic Image Capturing Device

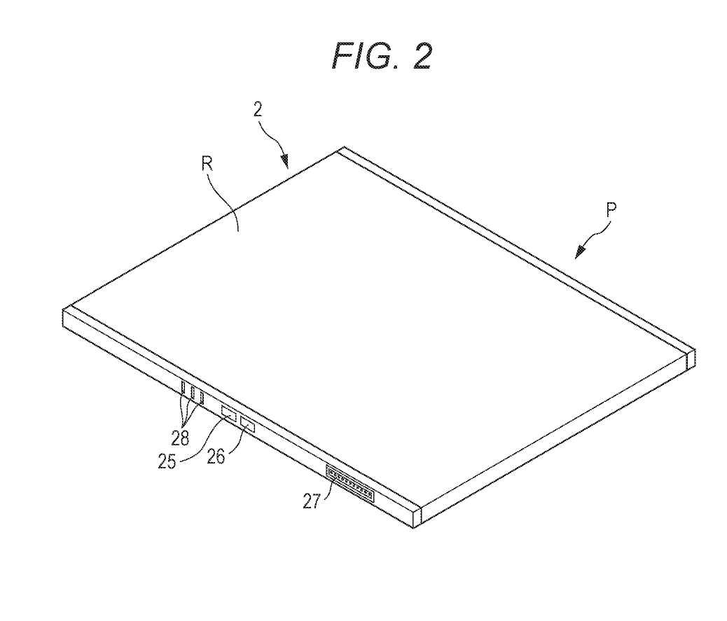

Here, the radiographic image capturing device P used in the radiographic image capturing system will be described. FIG. 2 is a perspective view illustrating an external appearance of the radiographic image capturing device.

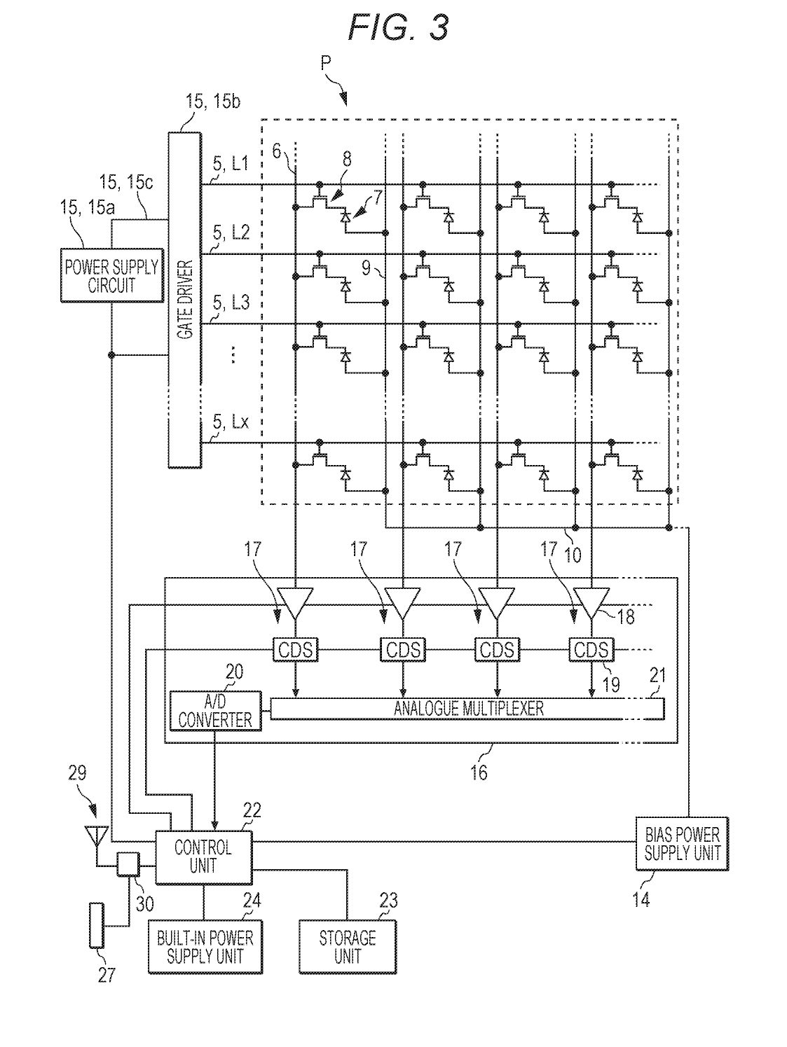

In the present embodiment, the radiographic image capturing device P has a housing 2 that stores therein the undermentioned radiation detection element 7 and the like. A power switch 25, a selection switch 26, the above-described connector 27, an indicator 28 and the like are arranged on one side surface of the housing 2. In addition, in the present embodiment, although illustration is omitted, an antenna 29 for performing wireless communications with the outside (refer to FIG. 3 described below) is provided, for example, on the other side surface of the housing 2. It should be noted that when wired communications with the outside are performed, a cable (not illustrated) is connected to the connector 27 to enable wired communications.

FIG. 3 is a block diagram illustrating an equivalent circuit of the radiographic image capturing device. As shown in FIG. 3, a plurality of radiation detection elements 7 are two-dimensionally arranged (in a matrix form) on a sensor substrate (not illustrated) of the radiographic image capturing device P. Each of the radiation detection elements 7 is configured to generate an electrical charge in response to the quantity of irradiated radiation. Corresponding bias lines 9 are connected to the radiation detection elements 7, and the bias tines 9 are connected to a connection line 10. Further, the connection line 10 is connected to a bias power supply unit 14, and the bias power supply unit 14 is configured to apply a reverse bias voltage to the radiation detection elements 7 through the bias lines 9 and the like.

Thin film transistors (hereinafter referred to as “TFT” (Thin Film Transistor)) 8 are connected to the radiation detection elements 7 respectively as switching elements. The TFTs 8 are connected to corresponding signal lines 6. In addition, a scan driving unit 15 is configured to switch, by a gate driver 15b, between an ON voltage and an OFF voltage that are supplied from a power supply circuit 15a through a wiring line 15c, and then to apply the voltage to scanning lines 5 (lines L1 to Lx). Moreover, when the ON voltage is applied to each of the TFTs 8 through the corresponding scanning line 5, the TFT 8 is brought into an ON state, which causes an electrical charge accumulated in the corresponding radiation detection element 7 to be emitted to the corresponding signal line 6. Meanwhile, when the OFF voltage is applied to each of the TFTs 8 through the corresponding scanning line 5, the TFT 8 is brought into an OFF state to interrupt the conduction between the corresponding radiation detection element 7 and the corresponding signal line 6, which causes an electrical charge generated in the radiation detection element 7 to be accumulated in the radiation detection element 7.

A read IC 16 has therein a plurality of read circuits 17, and the signal lines 6 are connected to the read circuits 17 respectively. In addition, in the case of read processing of reading the image data D, when an electrical charge is emitted from the radiation detection element 7, the electrical charge flows into the corresponding read circuit 17 through the corresponding signal line 6, and a corresponding amplifying circuit 18 outputs a voltage value in response to the amount of electrical charge that has been flown. Moreover, a correlation double sampling circuit (“CDS” in FIG. 3) 19 reads the voltage value, which has been output from the amplifying circuit 18, as the image data D based on an analogue value, and then outputs the voltage value to the downstream side. Subsequently, the output image data D is successively transmitted to an A/D converter 20 through an analogue multiplexer 21, and is then successively converted into the image data D based on a digital value by the A/D converter 20. The converted image data D is output to a storage unit 23, and is then successively saved therein.

A control unit 22 includes: a computer in which a CPU, a ROM, an RAM, an input-output interface and the like, which are not illustrated, are connected through buses; and a field programmable gate array (FPGA). The control unit 22 may be configured by a dedicated control circuit. In addition, a storage unit 23 that is formed of a static RAM (SRAM), a synchronous DRAM (SDRAM), a NAND flash memory or the like is connected to the control unit 22.

Moreover, a communication unit 30 that wirelessly or wiredly communicates with the outside through the antenna 29 or the connector 27 is connected to the control unit 22. Furthermore, a built-in power supply unit 24 such as a lithium ion capacitor is connected to the control unit 22. The built-in power supply unit 24 supplies required electric power to each functional part such as the scan driving unit 15, the read circuit 17, the storage unit 23, and the bias power supply unit 14.

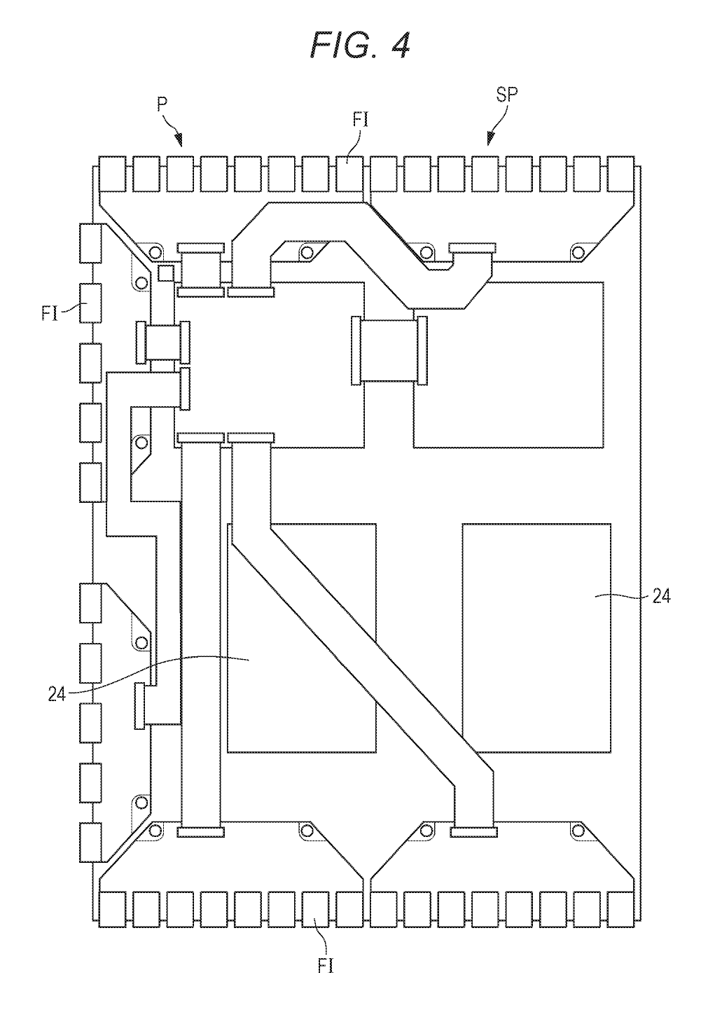

Incidentally, in the present embodiment, a sensor panel SP of the radiographic image capturing device P is formed of, for example, the sensor substrate on which the plurality of radiation detection elements 7 are formed. The sensor panel SP is stored in the housing 2 (refer to FIG. 2). In addition, FIG. 4 is a diagram illustrating a configuration example of the sensor panel, and is a diagram illustrating a back side view of the sensor panel SP (that is to say, viewed from the side opposite to the surface on which the radiation detection elements 7 are formed). In this manner, the front surface side (the radiation detection elements 7, etc.) and the rear surface side (the control unit 22, etc.) of the sensor panel SP are connected through a flexible circuit substrate F1. Moreover, the read IC 16 (refer to FIG. 3), and gate ICs (not illustrated) that constitute the gate driver 15b, are mounted on the flexible circuit substrate F1.

### Radiographic Image Capturing Stand

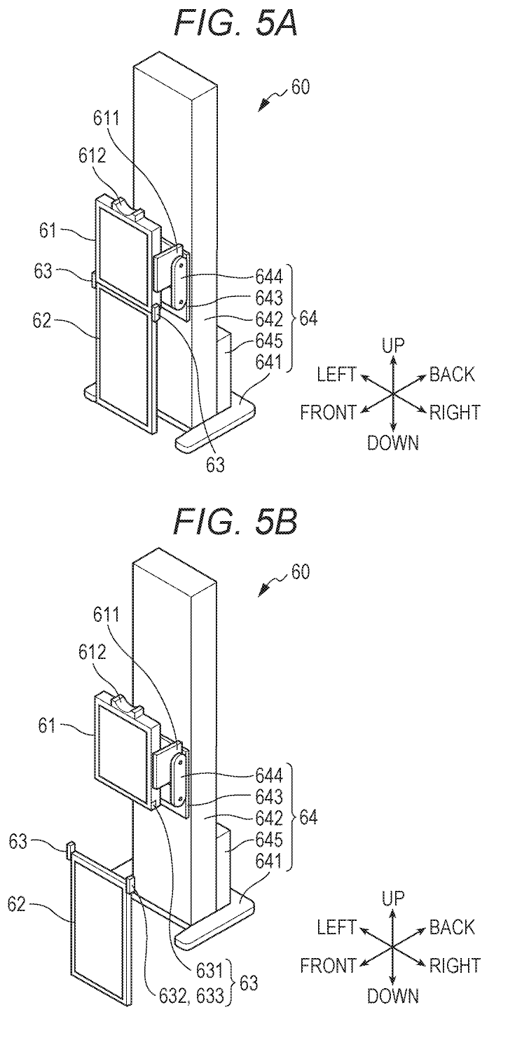

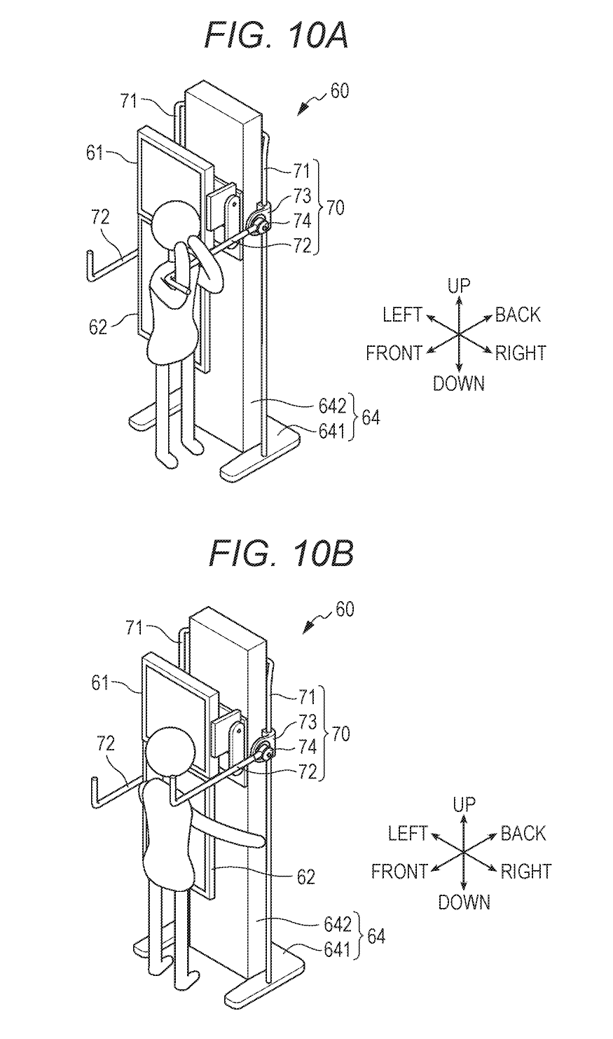

FIGS. 5A and 5B are perspective views each illustrating the radiographic image capturing stand 60. As shown in the figures, the radiographic image capturing stand 60 includes: a first holder 61 functioning as a first holding part that stores and holds the single radiographic image capturing device P1; a second holder 62 functioning as a second holding part that stores and holds a plurality of (for example, two) radiographic image capturing devices P2, P3 side by side; a coupler 63 that removably fixes and mounts the second holder 62 to the lower part of the first holder 61; a support stand 64 that supports the first holder 61; and a holding unit 70 (refer to FIG. 10) for, When a subject is subjected to radiographic image capturing, allowing the subject to hold on thereto.

It should be noted that up, down, left, right, front and back directions shown in FIGS. 5A and 5B are defined as up, down, left, right, front and back in the radiographic image capturing stand 60. In other words, one side in the longitudinal direction of the undermentioned support post 642 is defined as the “up” side, and the other side is defined as the “down” side; the side on which the support post 642 is provided with the first holder 61 is defined as the “front” side, and the side opposite thereto is defined as the “back” side; and when facing the first holder 61, the left-hand side is defined as the “left” side, and the right-hand side is defined as the “right” side.

### Radiographic Image Capturing Stand: First Holder

The first holder 61 is a housing that stores and holds the radiographic image capturing device P. In a basic holding posture, the first holder 61 stores and holds the radiographic image capturing device P in a state in which a radiation receiving surface of the radiographic image capturing device P is kept parallel to the vertical up-and-down direction, and is directed forward.

The front surface part of the first holder 61 is flat, and the first holder 61 is formed of a material, the radiation absorption rate of which is low. The right side surface of the first holder 61 is formed with a slot (illustration thereof is omitted) into which the radiographic image capturing device P is inserted. By inserting the radiographic image capturing device P from this slot up to a stop position, the radiographic image capturing device P can be held at a prescribed position.

It should be noted that the radiographic image capturing device P is stored and held in the first holder 61 in such a manner that the radiographic image capturing device P is kept parallel to the first holder 61 in both the up-and-down and right-and-left directions.

In addition, extending parts 611 to be supported by the support stand 64 are extended backward from the right and left side surfaces of the first holder 61 respectively (illustration of the extending part on the left side is omitted).

Moreover, the upper end part of the first holder 61 is provided with a chin support part 612 on which the neck of the subject at a standing position is placed so as to cause the chest to adhere to the front surface at the time of image capturing.

Furthermore, although described below, in the case of one-shot long-length image capturing, in order to enable the synthesization of a plurality of images captured by the plurality of radiographic image capturing devices P into one seamless image, it is necessary to adjacently arrange the plurality of radiographic image capturing devices P vertically side by side in such a manner that the tower end part of the sensor panel SP of the upper radiographic image capturing device P and the upper end part of the sensor panel SP of the lower radiographic image capturing device P overlap each other adjacently in ate front-and-back direction.

Therefore, the upper end part of the radiographic image capturing device P stored in the undermentioned second holder 62 protrudes outside, and the lower end part of the first holder 61 is formed with an opening (not illustrated) into which the upper end part of the radiographic image capturing device P is inserted.

### Radiographic Image Capturing Stand: Second Holder

The second holder 62 is a housing that stores and holds two radiographic image capturing devices P vertically side by side. In a basic holding posture in which the second holder 62 is fixed and mounted to the lower end part of the first holder 61, the second holder 62 stores and holds the two radiographic image capturing devices P in such a manner that radiation receiving surfaces of the two radiographic image capturing device P are kept parallel to the vertical up-and-down direction, and are directed forward.

The front surface part of the second holder 62 is fiat, and the second holder 62 is formed of a material, the radiation absorption rate of which is low. The second holder 62 has a length approximately twice that of the first holder 61, and the right side surface of the second holder 62 is formed with two slots vertically side by side into which the radiographic image capturing devices P (illustration thereof is omitted) are inserted. By inserting the radiographic image capturing devices P from these slots up to stop positions, the radiographic image capturing devices P can be held at prescribed positions.

Incidentally, in the case of the second holder 62, when each of the radiographic image capturing devices P is rectangular, the second holder 62 stores and holds the radiographic image capturing devices P in such a manner that the longitudinal directions of the housings 2 are kept parallel to the up-and-down direction inside the second holder 62 respectively.

In addition, the second holder 62 is coupled to the first holder 61 to carry out one-shot long-length image capturing. Therefore, two radiographic image capturing devices P are arranged vertically side by side inside the second holder 62 in such a manner that the lower end part of the sensor panel SP of the upper radiographic image capturing device P and the upper end part of the sensor panel SP of the lower radiographic image capturing device P overlap each other adjacently in the front-and-back direction.

Moreover, it is necessary to arrange the radiographic image capturing devices P in such a manner that the lower end part of the sensor panel SP of the radiographic image capturing device P inside the first holder 61 and the upper end part of the sensor panel SP of the upper radiographic image capturing device P inside the second holder 62 overlap each other adjacently in the front-and-back direction. Therefore, as described above, the second holder 62 is farmed with an opening (not illustrated) in such a manner that the upper end part of the radiographic image capturing device P that is stored in the upper side of the second holder 62 protrudes from the upper end part of the second holder 62 toward the outside. The protruded upper end part of the radiographic image capturing device P is adapted to be inserted into an opening formed in the lower end part of the first holder 61.

### Radiographic Image Capturing Stand: Coupler

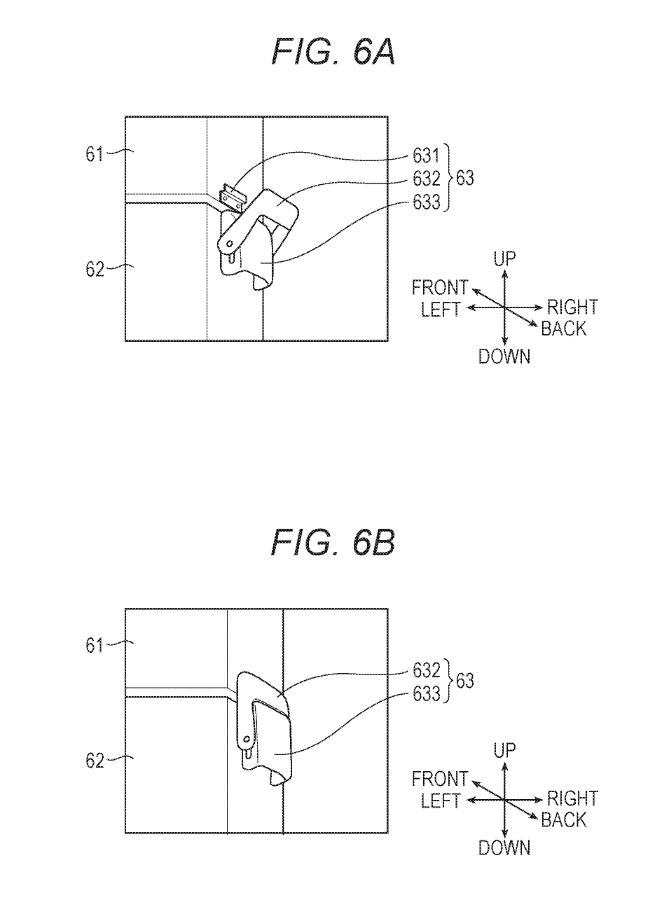

FIGS. 6A and 6B are perspective views each illustrating the coupler 63, FIG. 6A shows a non-coupled state, and FIG. 6B shows a coupled state.

The coupler 63 includes a pair of hook members 631 that is provided in the lower parts of the right and left side surfaces of the first holder 61 respectively, a pair of locking levers 632 that is provided in the upper parts of the right and left side surfaces of the second holder 62 respectively, and a pair of input levers 633. It should be noted that illustration of the hook member 631, the locking lever 632 and the input lever 633 on the left side surface is omitted.

The hook members 631 are hook-shaped members each having an end part directed upward.

A base end part of the locking lever 632 is tunably connected to the input lever 633 so as to allow the locking lever 632 to turn about a shaft along the front-and-back direction by the input lever 633, and a turning end part of the locking lever 632 is formed with a downward angled barb that can be hung on the upward angled hook member 631.

The upper end part of the input lever 633 is turnably supported by the upper part of the side surface of the second holder 62 so as to allow the input lever 633 to turn about a shaft along the front-and-back direction, and the lower end part of the input lever 633 turnably supports the base end part of the locking lever 632.

By turning the turning end part of the input lever 633 to lift the input lever 633 in a direction apart from the side surface of the second holder 62, the barb of the turning end part of the locking lever 632 supported by the input lever 633 can be lifted to a position above the hook member 631. Subsequently, when the turning end part of the input lever 633 is turned downward to a position in touch with the side surface of the second holder 62, the barb of the turning end part of the locking lever 632 moves downward, and is then coupled to the hook member 631. Thus, a state of FIG. 5B changes to a state of FIG. 5A, and consequently the second holder 62 can be fixedly coupled to the lower end part of the first holder 61.

Incidentally, the coupler 63 that includes the hook member 631, the locking lever 632 and the input lever 633 has a toggle mechanism, and is configured to exceed the dead center in a process of turning the input lever 633 downward. Therefore, even when the external force is applied in this state in the up-and-down direction in which the first holder 61 and the second holder 62 are spaced away from each other, the force is applied to the turning end part of the input lever 633 in a direction turning toward the side surface side of the second holder 62. As the result, the coupled state is not released unless the turning end part of the input lever 633 is artificially turned in a direction spaced away from the side surface side of the second holder 62.

It should be noted that the coupler 6:3 is not limited to the above-described configuration, and therefore it is possible to use all means that enable coupling of the second holder 62 to the first holder 61 with the constant posture maintained without causing wobbling, and that are arbitrarily removable as necessary

### Radiographic Image Capturing Stand: Support Stand

The support stand 64 includes: a tabular base plate 641 that is located in the lower part, and supports the overall configuration of the radiographic image capturing stand 60; the support post 642 that is erected from the upper surface of the base plate 641 in the vertical up-and-down direction; a holder lifting and lowering body 643 that moves up and down along the support post 642 to enable height adjustment; a pair of support arms 644 that is provided on the holder lifting and lowering body 643, and supports the first holder 61; a counter weight (not illustrated) for adjusting the weight balance of the first holder 61; and an electrical component part 645.

The holder lifting and lowering body 643 is movably supported along the up-and-down direction through a slide guide (not illustrated) with respect to the front surface part of the support post 642, and is capable of fixing a position at an arbitrary height by well-known fixing means (not illustrated), In addition, the holder lifting and lowering body 643 is tunably and pivotally supported by a support shaft along the front-and-back direction with respect to the front surface part of the support post 642. The holder lifting and lowering body 643 is capable of fixing a direction by the above-described well-known fixing means at an arbitrary inclination angle.

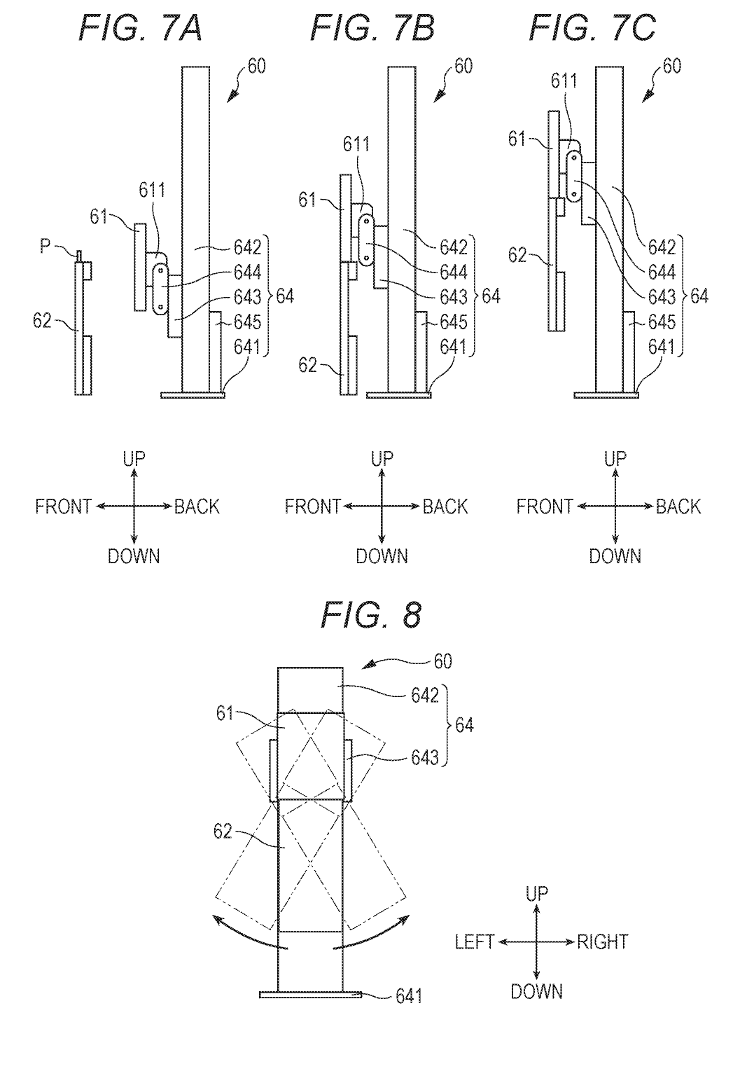

Therefore, as shown in FIGS. 7A to 7C, respective heights of the first and second holders 61, 62 can be adjusted as appropriate in the single image capturing state or in the long-length image capturing state.

In addition, at the time of radiographic image capturing, it may be difficult to support the upright posture of a subject due to a physical state of the subject. However, as shown in FIG. 8, configuring the first holder 61, or the first holder 61 and the second holder 62, to be capable of turning about the shaft in the front-and-back direction by the holder lifting and lowering body 643 enables image capturing in a state of being inclined at an arbitrary inclination angle.

In addition, the first holder 61 is supported by the pair of support arms 644. The pair of support arms 644 (the left-side support arm 644 is not illustrated) has the respective base end parts that are supported so as to turn about a shaft along the right-and-left direction on the right and left side surfaces of the holder lifting and lowering body 643 respectively. The turning end parts are turnably connected to the end parts of the pair of extending parts 611 of the first holder 61 so as to turn about shafts along the right-and-left direction respectively.

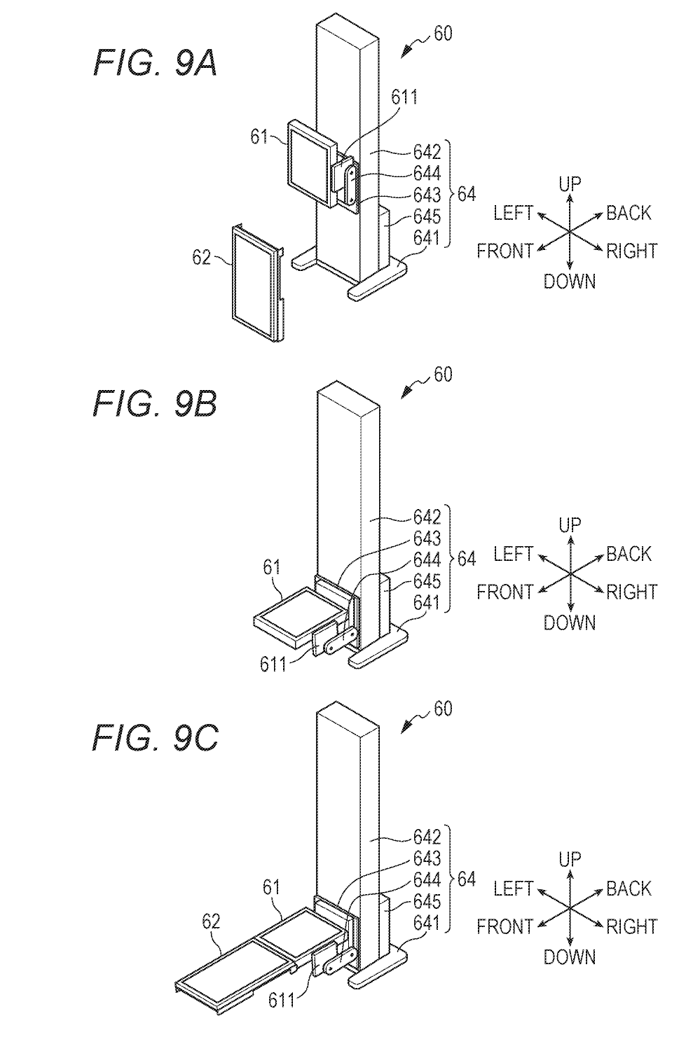

As the result, as shown in FIG. 9A, by tilting the pair of support arms 644 forward from a state in which the pair of support arms 644 stands up, the first holder 61 (and the second holder 62) can be moved forward and spaced away from the holder lifting and lowering body 643.

This enables the front surface (radiation incident plane) of the first holder 61 (and the second holder 62) to tilt about the shaft along the right-and-left direction without causing the support post 642 and the holder lifting and lowering body 643 to interfere with the first holder 61.

Moreover, as shown in FIGS. 9B and 9C, the front surface (radiation incident plane) of the first holder 61 (and the second holder 62) can be directed vertically upward. Therefore, it is also possible to perform image capturing in a state in which a subject lies on the radiation incident plane of the first holder 61 (and the second holder 62).

In the radiographic image capturing stand 60, components to be supported are concentrated on the front surface side of the support post 642, and therefore in order to prevent the radiographic image capturing stand 60 from falling forward, the radiographic image capturing stand 60 is fixed to a floor surface by using anchors through the tabular base plate 641. As the result, even when the subject places his/her weight on the first and second holders 61, 62, the radiographic image capturing stand 60 can be prevented from falling forward.

In addition, as an alternative to the above-described anchors, the rear part of the radiographic image capturing stand 60 may be equipped with a weight to maintain the weight balance, thereby preventing the radiographic image capturing stand 60 from falling forward.

### Radiographic Image Capturing Stand: Holding Unit

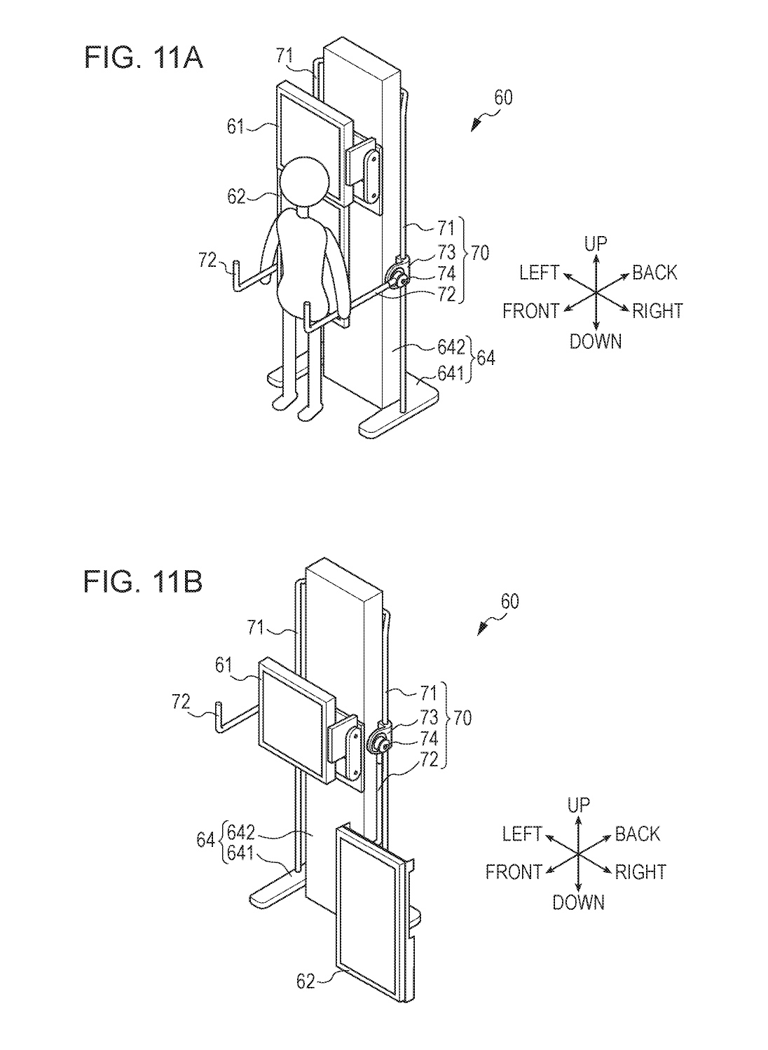

FIGS. 10A to 11B are perspective views each illustrating a use state of the holding unit 70. As shown in the figures, the holding unit 70 includes: first holding bars 71 functioning as grips that are erected on the base plate 641 as a pair of right and left grips arranged on both the right and left sides of the support post 642 respectively; second holding bars 72 functioning as a pair of right and left grips that are mounted to the first holding bars 71 respectively; height adjusting mechanisms 7:3 that adjust heights of the second holding bars 72 respectively; and posture adjusting mechanisms 74 that adjust directions of the second holding bars 72 respectively.

It should be noted that the first holding bars 71, the second holding bars 72, the height adjusting mechanisms 73, and the posture adjusting mechanisms 74, which constitute the holding unit 70, are all provided as a pair of right and left components, and thus have symmetric structures. Therefore, description will be given exclusively on the right components.

The right and left first holding bars 71 are each formed as a rod-shaped body along the up-and-down direction, and are disposed adjacently to both the right and left sides of the support post 642 respectively. The respective upper end parts of the right and left first holding bars 71 are connected to the support post 642.

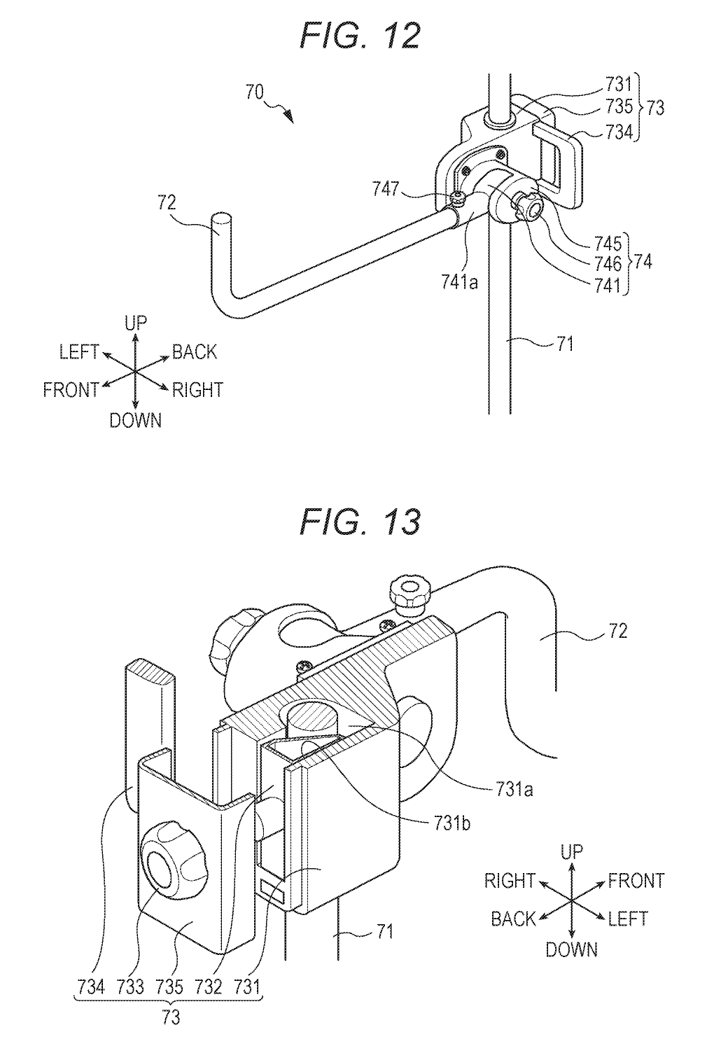

FIG. 12 is a perspective view illustrating the second holding bar 72, the height adjusting mechanism 73, and the posture adjusting mechanism 74 on the right side,

As shown in the figure, the second holding bar 72 is a rod-shaped body having a length shorter than that of the first holding bar 71, The end part of the second holding bar 72 is bent substantially in the shape of L. In addition, the base end part of the second holding bar 72 is supported by the first holding bar 71 through the height adjusting mechanism 73 and the posture adjusting mechanism 74.

FIG. 13 is a perspective view illustrating an internal structure of the height adjusting mechanism 73 that is partially cut off. As shown in FIGS. 12 and 13, the height adjusting mechanism 73 includes: a support block 731 into which the first holding bar 71 is inserted in a penetrated state; a wedge-shaped member 732 that is pressure-welded to the first holding bar 71 inside the support block 731; a fastening screw 733 that moves the wedge-shaped member 732 along the pressure welding direction; and a handle 734 that is handled at the time of height or posture adjustment.

The support block 731 is formed with a through hole 731a along the up-and-down direction, through which the first holding bar 71 can be loosely inserted. In addition, the support block 731 is formed with a guide hole 731b that movably guides the wedge-shaped member 732 in the front-and-back direction from the back end surface of the support block 731 to the through hole 731a.

In addition, a bracket 735, which is formed with a threaded screw hole through which the fastening screw 733 is engaged, is fixed and mounted to the rear end surface of the support block 731. The end part of the fastening screw 733 is rotatably connected to the rear end surface of the wedge-shaped member 732 contained in the guide hole 731b.

The fastening screw 733 has a knob-shaped head that can be manually rotated, and that is exposed outside the bracket 735. Rotating the head enables the wedge-shaped member 732 to move backward and forward.

When the wedge-shaped member 732 is moved forward by the fastening screw 733, an inclined surface of the end part thereof is pressed on the outer peripheral surface of the first holding bar 71. In addition, by pressing the outer peripheral surface of the first holding bar 71 on the inner peripheral surface of the through hole 731a, the first holding bar 71 can be relatively fixed to the support block 731 by friction. Moreover, when the fastening screw 733 is loosened, a state of being fastened by friction can be released. Consequently, the second holding bar 72 can be moved up and down through the support block 731.

Therefore, by the manual operation of the fastening screw 733, the second holding bar 72 can be adjusted to an arbitrary height to fix the second holding bar 72 at the height.

In addition, by loosening the fastening screw 733, the height adjusting mechanism 73 allow the second holding bar 72 to turn about the first holding bar 71, thereby enabling an extending direction of the second holding bar 72 to be adjusted.

Moreover, in contrast, When it is not necessary to turn the second holding bar 72 about the first holding bar 71 for adjustment, the height adjusting mechanism 73 may have a structure in which turning is not allowed by forming the first holding bar 71 with a spline groove along the up-and-down direction, and by forming the support block 731 with a spline nut structure.

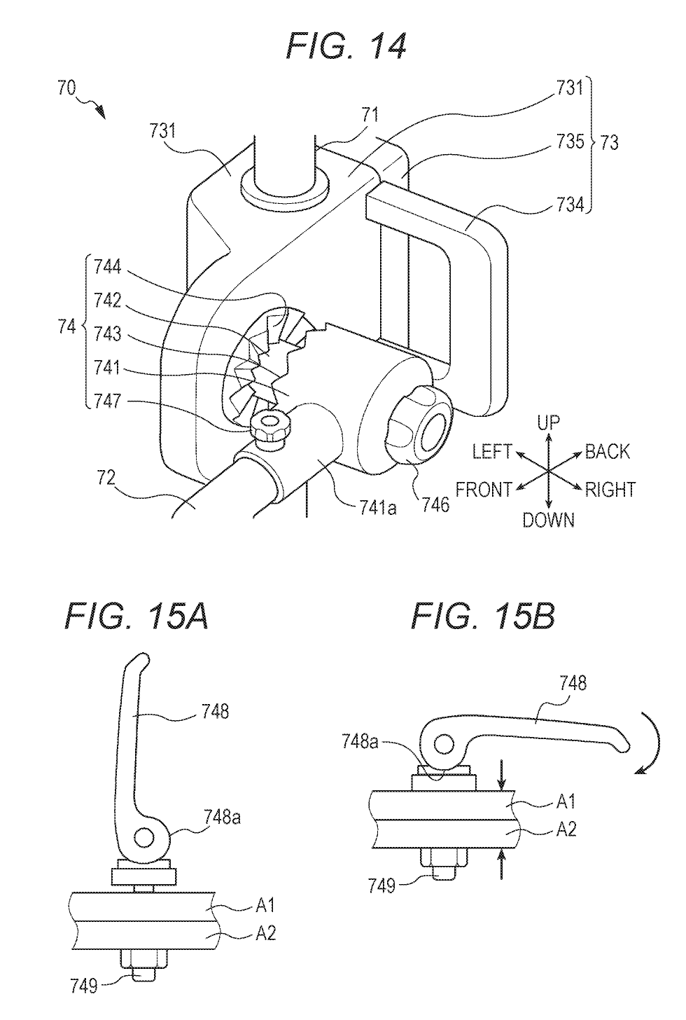

FIG. 14 is a perspective view illustrating an internal structure of the posture adjusting mechanism 74, the configuration of which is partially excluded. As shown in FIGS. 12 and 14, the posture adjusting mechanism 74 includes: a holding body 741 that holds the second holding bar 72; a support shaft 742 that is mounted to the support block 731 described above, and that turnably supports the holding body 741 so as to allow the holding body 741 to turn about the shaft along the right-and-left direction; engaging teeth 743, 744 that are formed between the support block 731 and the holding body 741; a spring (not illustrated) that applies pressure in a direction in which the engaging teeth 743, 744 engaged with each other; a cover 745 that holds the holding body 741 so as to prevent the holding body 741 from falling from the support shaft 742; a first fastening screw 746 that fastens and holds the second holding bar 72 and the holding body 741 to prevent the second holding bar 72 and the holding body 741 from turning about the support shaft 742; and a second fastening screw 747 that fastens and holds the base end part of the second holding bar 72.

The support shaft 742 is extended from the right end surface of the support block 731 toward the right side. In addition, the holding body 741 has a substantially cylindrical shape, and is capable of turning about the support shaft 742 with the support shaft 742 inserted therein. The outer periphery of the holding body 741 is integrally formed with a cylindrical part 741a that is extended outward in a radial direction that defines the support shaft 742 as the center thereof. The base end part of the second holding bar 72 is inserted and held in the cylindrical part 741a.

The second fastening screw 747 is screwed in a threaded screw hole that is penetratingly formed from the outer peripheral surface of the cylindrical part 741a to the inside. The end part of the second fastening screw 747 enters a circumferential groove (not illustrated) that is formed in the outer periphery of the base end part of the second holding bar 72. In addition, a manually rotatable knob-shaped head of the second fastening screw 747 is exposed outside the cylindrical part 741a. Rotating the head enables the end part to move forward and backward.

Therefore, manually rotating the second fastening screw 747 to move the end part forward causes the end part to be pressure-welded on the bottom part of the circumferential groove in the base end part of the second holding bar 72. Consequently, the second holding bar 72 is fixed to the cylindrical part 741a so as not to cause the rotation about the shaft along the longitudinal direction of the second holding bar 72.

Moreover, when the second fastening screw 747 is loosened, a pressure welding state of the end part is released, and consequently the second holding bar 72 can be rotated about the shaft along the longitudinal direction of the second holding bar 72.

Therefore, the second holding bar 72 is rotated about the shaft along the longitudinal direction of the second holding bar 72 to adjust the bending part at the end of the second holding bar 72 to an arbitrary direction, and subsequently the bending part can be fixed by tightening the second fastening screw 747.

in addition, the holding body 741 can be slidingly moved along the support shaft 742 in the right-and-left direction, The engaging teeth 744 that are convex toward the right side along the circumference are formed on the right end surface of the support block 731 and around the support shaft 742. The engaging teeth 743 that are convex toward the left side along the circumference are also formed on the left end surface of the holding body 741.

Moreover, the spring in the holding body 741 presses the holding body 741 to the left side to cause the engaging teeth 743, 744 to engage with each other.

Therefore, when the second holding bar 72 is turned about the support shaft 742 to carry out angle adjustment, it is necessary to apply the constant force against the spring in such a manner that the holding body 741 is slidingly moved further than the depth at which the engaging teeth 743, 744 engage with each other. When the force exceeds the holding force by the spring, an angle of the second holding bar 72 can be adjusted about the support shaft 742 in units of angle formed by two adjacent teeth of the engaging teeth 743, 744.

The first fastening screw 746 penetrates the right end surface of the holding body 741, and is screwed into the threaded screw hole (not illustrated) with which the support shall 742 is provided. In addition, a manually rotatable knob-shaped head of the first fastening screw 746 is exposed from the right end surface of the holding body 741. Manually rotating the head to fasten the first fastening screw 746 enables the holding body 741 to be secured so as to prevent the holding body 741 from slidingly moving rightward with respect to the support shaft 742.

Therefore, when an angle of the second holding bar 72 about the support shaft 742 is adjusted, the first fastening screw 746 is loosened, the second holding bar 72 is turned in units of angle formed by two adjacent teeth of the engaging teeth 743, 744 to arbitrarily adjust the angle, and the first fastening screw 746 is then fastened, thereby enabling the second holding bar 72 to be fixed at the angle after the adjustment.

It should be noted that as an alternative to the first fastening screw 746, as shown in FIGS. 15A and 15B, there may be employed a configuration composed of: a toggle-type locking lever 748 having a base end part that is integrally provided with a cam part 748a; and a pin 749. In this configuration, one end part of the pin 749 is provided with a turning support shaft of the toggle-type locking lever 748. Turning the toggle-type locking lever 748 from a state of FIG. 15A to a state of FIG. 15B enables a member A1 that comes in contact with the cam part 748a to be pressed toward the side of a member A2 connected to the other end of the pin 749.

Therefore, by connecting the other end of the pin 749 to the support shaft 742 in such a manner that the cam part 748a of the toggle-type locking lever 748 presses the right end surface of the holding body 741 leftward, the turning operation of turning the toggle-type locking lever 748 enables a state in which an angle of the second holding bar 72 about the support shaft 742 can be adjusted and a state in which the angle is fixed to be easily switched.

In this manner, the holding unit 70 is provided with the first holding bar 71, the second holding bar 72, the height adjusting mechanism 73, and the posture adjusting mechanism 74. Therefore, as shown in FIG. 10A, radiographic image capturing of a subject can be performed while the subject is holding on to the second holding bar 72, the height and posture of Which have been properly adjusted, or as shown in FIG. 10B, radiographic image capturing of a subject can be performed while the subject is holding on to the first holding bar 71.

Moreover, as shown in FIG. 11A, radiographic image capturing of a subject can also be performed while the subject is holding on to the pair of second holding bars 72 that is horizontally extended.

Furthermore, when the radiographic image capturing stand 60 is installed in a limited narrow space, with the result that it is required to disconnect the second holder 62 from the first holder 61, and then to put the second holder 62 in either the right or left direction, as shown in FIG. 11B, tilting the second holding bar 72 located in such a direction downward enables the second holder 62 to be easily moved.

### Moving Auxiliary Tool

In the case of single image capturing that uses only the first holder 61, the second holder 62 is disconnected from the first holder 61, and is then transported to a predetermined retracted position so as not to hinder image capturing, The second holder 62 is large sized, and is heavy in weight too. Therefore, the second holder 62 is transported by a dedicated moving auxiliary tool 80.

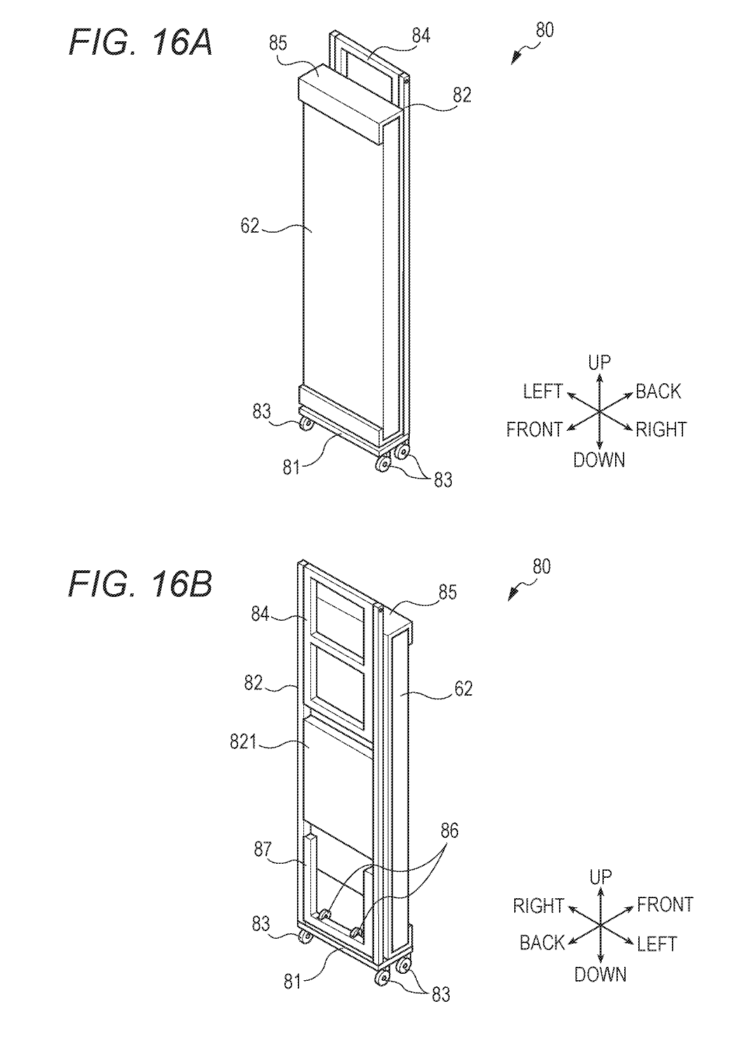

FIG. 16A is a perspective view illustrating the front surface side of the moving auxiliary tool 80; and FIG. 16B is a perspective view illustrating the back surface side. In the description of the moving auxiliary tools 80, as shown in FIGS. 16A and 16B, in a state in which the radiation incident plane of the second holder 62 held by the moving auxiliary tool 80 is aligned to the vertical plane, the front surface side perpendicular to the radiation incident plane is defined as the “front” side of the moving auxiliary tool 80, the back surface side is defined as the “back” side, one end side in the longitudinal direction of the second holder 62 is defined as the “up” side, the other end side is defined as the “down” side. When facing the first holder 61, the left-hand side is defined as the “left” side, and the right-hand side is defined as the “right” side.

The moving auxiliary tool 80 includes: a bottom frame 81 that supports the whole on the bottom; a mainframe 82 that is erected from the rear end part of the bottom frame 81; four casters 83 that are provided at four corners of the bottom surface of the bottom frame 81 respectively; a movable handle 84 that is provided at the upper end part of the mainframe 82; a holding frame 85 that holds the second holder 62; and a support leg part 87 provided with an auxiliary caster 86.

The bottom frame 81 is a rectangular frame having a right-to-left width that is a little wider than that of the second holder 62, and having a front-to-back width that is a little wide.

The mainframe 82 is a rectangular frame having a right-to-left width similar to that of the second holder 62, and having a height that is a little higher than that of the second holder 62. The central part in the up-and-down direction of the frame is provided with a support plate 821 that tunably supports the holding frame 85 so as to allow the holding frame 85 to turn about the shaft along the front-and-back direction. In addition, the upper end part of the mainframe 82 is separated, and a rectangular frame-shaped movable handle 84 is arranged between the upper end parts in a state in which the movable handle 84 is housed in the mainframe 82. The movable handle 84 is turnably supported by the mainframe 82 so as to allow the movable handle 84 to turn about a shaft along the right-and-left direction.

The radiographic image capturing device P that is stored in the first holder 61 or the second holder 62 has a function of, when the radiation incident plane is irradiated with a radiation ray, detecting the radiation ray, and then automatically generating a radiographic image. Therefore, the radiographic image capturing device P is capable of performing radiographic image capturing even in a state in which the second holder 62 is separated from the radiographic image capturing stand 60.

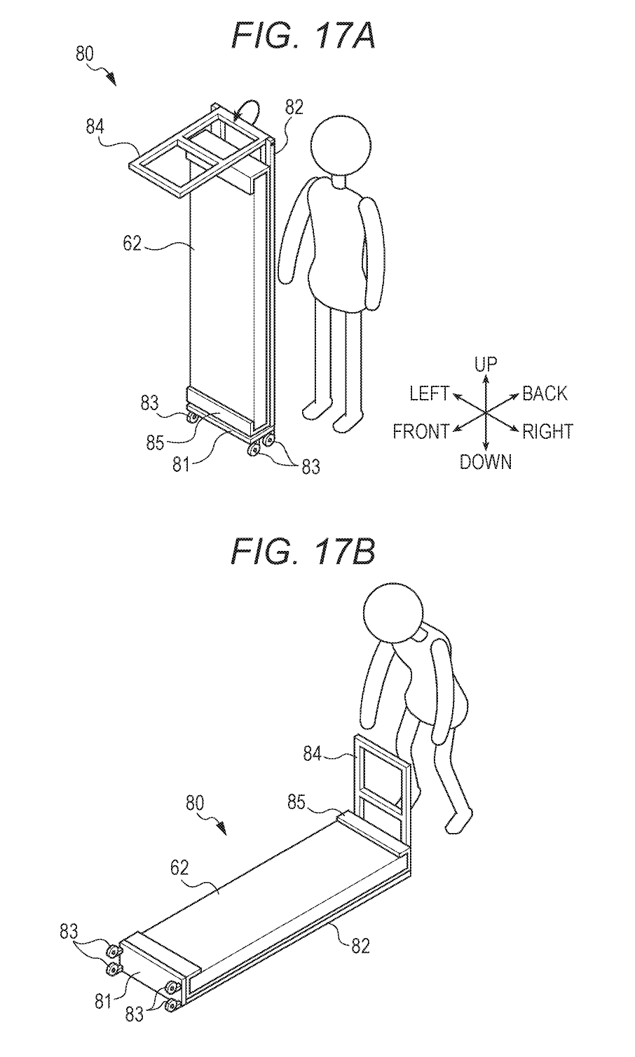

Therefore, as shown in FIGS. 17A and 17B, it is also assumed that radiographic image capturing is performed in a state in which the second holder 62 is held in the moving auxiliary tool 80.

The above-described movable handle 84 is suitable for a case where the second holder 62 is laid down to bring the second holder 62 into a horizontal state, and image capturing of a subject in a lying state is performed.

In other words, as shown in FIG. 17A, the lower end part of the movable handle 84 is turned forward, and as shown in FIG. 17B, the work of laying the whole down with the lower end part of the moving auxiliary tool 80 used as a fulcrum is performed to bring the moving auxiliary tool 80 into a horizontal state. In this case, the movable handle 84 is brought into a state of being erected upward, and therefore an operator is not required to stoop down, which enables a reduction in workload.

In addition, the support leg part 87 is arranged at a position in proximity to the lower end part of the mainframe 82 in a state in which the support leg part 87 is housed in the mainframe 82.

The support leg part 87 is a frame-shaped body that is integrally formed by: a pair of right and left leg parts along the up-and-down direction; and a crossbar that connects between the lower end parts of the leg parts. Both the right and left ends of the crossbar extending in the right-and-left direction are provided with the auxiliary casters 86 respectively.

Moreover, the upper end parts of the support leg part 87 are turnably supported by the mainframe 82 so as to allow the support leg part 87 to turn about shafts along the right-and-left direction.

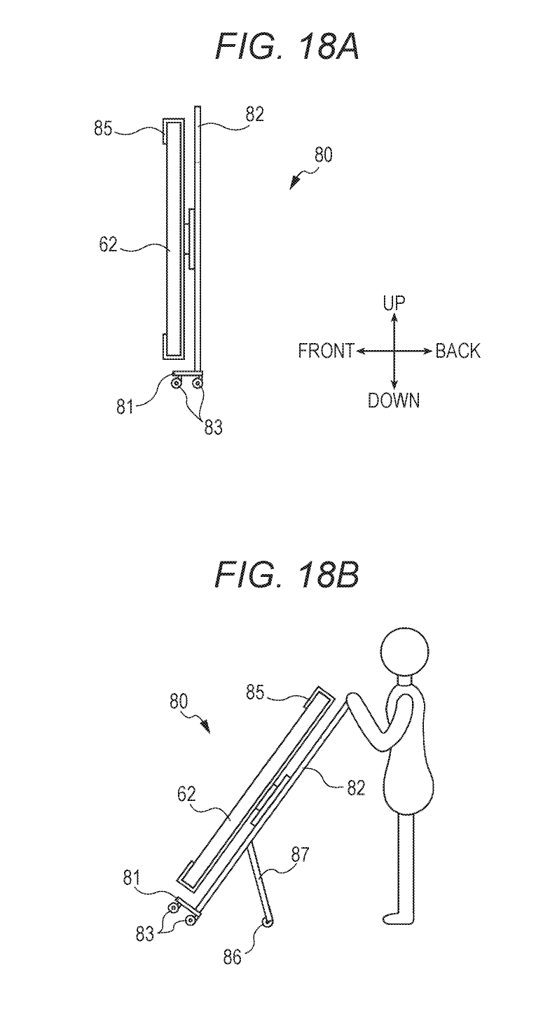

As shown in FIG. 18A, it is difficult to perform the work of transporting the second holder 62 with the mainframe 82 perpendicularly erected. Therefore, as shown in FIG. 18B, tilting the upper end part of the mainframe 82 backward makes the movement of the mainframe 82 easier. In this case, by tilting the lower end part of the support leg part 87 backward to bring the auxiliary caster 86 provided on the lower end part into contact with the ground, the tilting posture of the mainframe 82 is stabilized, thus enabling more stable transportation.

The holding frame 85 is integrally formed by: a flat plate having a size that is substantially equal to that of the back surface of the second holder 62; and U-shaped cross sectional frame parts that are provided in the upper and lower end parts of the flat plate respectively. The U-shaped cross-sectional frame part provided in the upper end part has an opening facing downward, and the U-shaped cross-sectional frame part provided in the lower end part has an opening facing upward. Such a shape allows the second holder 62 to be slidingly inserted into the holding frame 85 from either the right or left side. After the insertion, a lock mechanism (not illustrated) enables the second holder 62 to be securely held by the upper and lower frame parts.

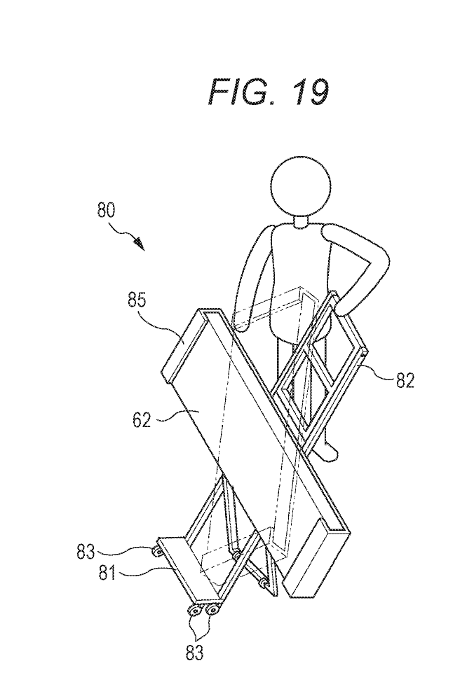

As described above, the holding frame 85 is turnably supported by the mainframe 82 so as to allow the holding frame 85 to turn about a shaft along the front-and-back direction. Therefore, as shown in FIG. 19, the second holder 62 can be turned in a state in which the front face of the second holder 62 that is being held faces forward.

Therefore, the second holder 62 can be tilted in such a manner that the longitudinal direction of the second holder 62 is kept horizontal. For example, a subject who lies in a bed is irradiated with a radiation ray from the side, and the radiation ray passing through the subject is then detected, thereby enabling radiographic image capturing of the lying subject by the lateral irradiation.

### Radiographic Image Capturing Operation

The radiographic image capturing operation in the above-described radiographic image capturing system 50 will be described.

The radiographic image capturing devices P1 to P3, which are stored in the first and second holders 61, 62 of the radiographic image capturing stand 60, are provided with respective dongles, each of which stores individual identification information.

The identification information of each of the radiographic image capturing devices P1 to P3 is registered, in the console C, by being associated with information indicating where each of the radiographic image capturing devices P1 to P3 is stored, that is to say, any one of the first holder 61, and upper and lower slots of the second holder 62.

Meanwhile, the radiographic image capturing stand 60 is provided with a state detection unit that detects either a state in which the second holder 62 is not connected to the first holder 61 (a state of being equipped with only the first holder 61) or a state in which the second holder 62 is connected to the first holder 61.

This state detection unit can be realized by, for example, a micro switch provided on the bottom of the first holder 61, an optical element that detects the connection by being shielded by the second holder 62, or the like.

In addition, the detection result obtained by the state detection unit is transmitted to the console C through the relay 54 by wireless communication or wired communication.

When single image capturing is performed by using the above-described radiographic image capturing system 50, the first holder 61 storing the radiographic image capturing device P1, which is inserted through a slot, is used with the second holder 62 disconnected.

Subsequently, a subject is subjected to position adjustment and the like, and the subject is then exposed to a radiation ray from the radiation irradiation device 52. As the result, the radiographic image capturing device P1 detects the radiation ray, generates radiographic image data, and then transmits the radiographic image data to the console C together with identification information.

The console C subjects the radiographic image data obtained from the radiographic image capturing device P1 to predetermined image processing, and then stores the processed image data in the storage unit Cb with the image data associated with identification data of the radiographic image capturing device P1.

In addition, when long-length image capturing (one-shot long-length image capturing) is performed, the second holder 62 storing the radiographic image capturing devices P1 to P3, which are inserted through individual slots, is connected to the first holder 61. Moreover, the state detection unit detects a connection state in which the second holder 62 is connected to the first holder 61. The connection state is then transmitted to the console C.

Subsequently, a subject is subjected to position adjustment and the like, and the subject is then exposed to a radiation ray from the radiation irradiation device 52. As the result, each of the radiographic image capturing devices P1 to P3 detects the radiation ray, generates radiographic image data, and then transmits the radiographic image data to the console C together with identification information.

The console C identifies, from accompanying identification information, how the pieces of received radiographic image data are associated with the radiographic image capturing devices P1 to P3, and further how the pieces of received radiographic image data are associated with three slots provided in the first and second holders 61, 62. Subsequently, the console C connects the pieces of radiographic image data together in the order of arrangement at the time of image capturing to synthesize the pieces of radiographic image data into single long-length radiographic image data. Such synthesization is required to carry out adjustment processing of adjusting differences in size by scaling, the differences being caused by differences in distance from the radiation irradiation device among the radiographic image capturing devices P1 to P3, and image processing of removing structure reflection components caused by the radiographic image capturing device P arranged on the front side.

Further, the long-length radiographic image data synthesized into one is also subjected to the same image processing as that of usual radiographic image data, and the processed long-length radiographic image data is then stored in the storage unit Cb with the processed long-length radiographic image data associated with identification data of the radiographic image capturing devices P1 to P3 and arrangement information thereof.

### Technical Effects of First Embodiment

The above-described radiographic image capturing stand 60 of the radiographic image capturing system 50 is capable of switching between a single image capturing state in which image capturing is performed with the single radiographic image capturing device P1 held by the first holder 61 with respect to one exposure to a radiation ray, and a long-length image capturing state in which image capturing is performed with the three radiographic image capturing devices P1 to P3 held by the first and second holders 61, 62 and arranged in a line with respect to one exposure to a radiation ray.

Therefore, in the case of image capturing of a child, or a subject with small stature, who cannot put the chin on a high place, or a subject using a wheelchair, who needs to maintain a sufficient space under the radiographic image capturing device P, single image capturing can be performed by using the first holder 61. When long-length image capturing is required, long-length image capturing can be performed by using the first holder 61 and the second holder 62.

Moreover, the coupler 63 fixedly connects the second holder 62 to the first holder 61 to hold the second holder 62. Therefore, even in the case of long-length image capturing, the second holder 62 is kept stable, and a constant direction can be maintained, thereby enabling excellent long-length image capturing to be achieved

The above-described feature enables the radiographic image capturing stand 60 to be provided that eliminates the need for individually preparing a radiographic image capturing stand used exclusively for single image capturing, and a radiographic image capturing stand used exclusively for long-length image capturing, and that is capable of realizing excellent single image capturing and excellent long-length image capturing even in the case of a narrow space.

In addition, the radiographic image capturing stand 60 is provided with; the first holder 61 that stores and holds the single radiographic image capturing device P1; the second holder 62 that stores and holds the two radiographic image capturing devices P2, P3 side by side; and the coupler 63 that removably fixes and mounts the second holder 62 to the lower part of the first holder 61. Therefore, the coupler 63 enables easy and quick switching between a single image capturing state and a long-length image capturing state, which enables an improvement in the workability at the time of image capturing to be achieved.

Moreover, the radiographic image capturing stand 60 is provided with the holding unit 70 that is provided with the first holding bar 71 and the second holding bar 72. Therefore, image capturing can be performed in a state of being assisted by the holding bars 71, 72, This enables a reduction in load at the time of image capturing even in the case of a subject who has difficulty in having a constant posture and in maintaining the posture. Further, this makes it possible to perform image capturing in various kinds of postures.

### Others

It should be noted that although the case where the two radiographic image capturing devices P are stored in the second holder 62 has been presented, the number of radiographic image capturing devices P to be stored may be one, or three or more. When three or more radiographic image capturing devices P are stored, it is preferable that the radiographic image capturing devices P be arranged in a line along the longitudinal direction of the radiographic image capturing device P.

In addition, although the case where the second holder 62 is removably fixed and mounted to the lower part of the first holder 61 has been presented, the configuration is not limited to this case, The second holder 62 may be configured to be removably fixed and mounted to an outer edge part of the first holder 61, that is to say, to the upper end part, the left end part, the right end part or the like.

### Second Embodiment

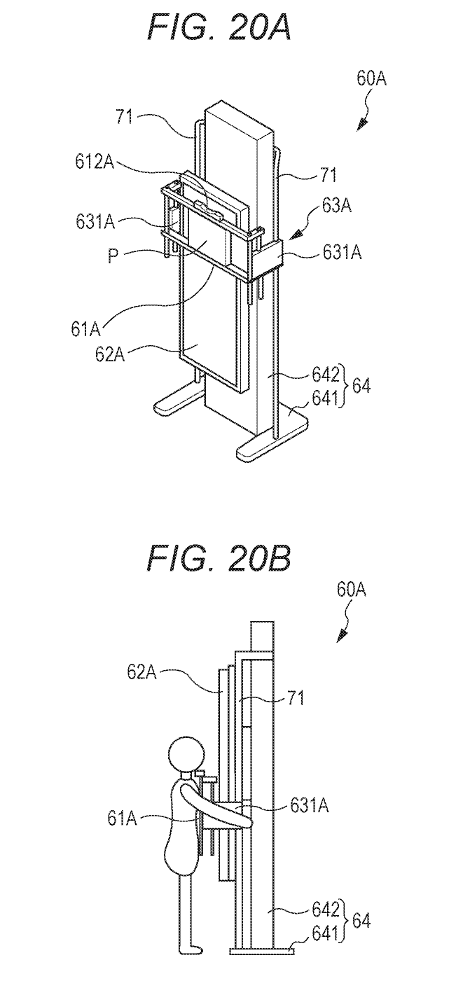

As a second embodiment of the present invention, another type of radiographic image capturing stand 60A will be described on the basis of FIGS. 20A and 20B. The radiographic image capturing system 50 is equipped with this radiographic image capturing stand 60A as an alternative to the radiographic image capturing stand 60 described above.

It should be noted that with respect to the radiographic image capturing stand 60A, identical reference numerals are used for components identical to those of the radiographic image capturing stand 60, and overlapping explanation will be omitted,

The radiographic image capturing stand 60A includes: a first holder 61A functioning as a first holding part that stores and holds the single radiographic image capturing device P; a second holder 62A functioning as a second holding part that stores and holds a plurality of (for example, three) radiographic image capturing devices P side by side; a coupler 63A that removably fixes and mounts the first holder 61A to the front side of the second holder 62A; a support stand 64 that supports the second holder 62A; and a holding unit 70 (only a first holding bar 71 is illustrated) for, when a subject is subjected to radiographic image capturing, allowing the subject to hold on thereto.

The second holder 62A differs from the above-described second holder 62 in that the three radiographic image capturing devices P are vertically stored side by side, and that the second holder 62A is directly supported by the support stand 64. Other than the above points of difference, the second holder 62A is the same as the above-described second holder 62. The second holder 62A is provided with three slots (not illustrated), and stores three radiographic image capturing devices P therein. Therefore, in the case of long-length image capturing, the second holder 62A alone is capable of performing image capturing.

In addition, since the second holder 62A stores the three radiographic image capturing devices P, it is not necessary to store the three radiographic image capturing devices P with the upper end part of the radiographic image capturing device P, which is stored in the uppermost part, protruded from the upper end part of the holder toward the outside as with the second holder 62. All of the radiographic image capturing devices P are stored inside the holder.

The first holder 61A is a rectangular frame body that holds the radiographic image capturing device P in the central part in the frame. The first holder 61A holds the radiographic image capturing device P with the radiation receiving surface of the radiographic usage capturing device P directed forward to be exposed to the outside.

In addition, the upper central part of the first holder 61A is equipped with a chin support part 612A.

The coupler 63A is composed of a pair of connecting arms 631A that is extended backward from both the right and left ends of the first holder 61A respectively.

The rear end parts on the respective opposite surfaces of the pair of connecting arms 631A are formed with semicircular grooves along the up-and-down direction respectively. The first holding bars 71 fit into the grooves. By fitting the first holding bars 71 into the grooves of the right and left connecting arms 631A, the first holder 61A can be connected to the front surface side of the second holder 62A.

It should be noted that one ends of the pair of connecting arms 631A are provided with respective attaching/detaching structures that use fixing parts each having constant elasticity, The elastic deformation of the fixing parts enables the first holder 61A to be attached to and detached from the pair of first holding bars 71.

In addition, the attaching/detaching structures are adapted to connect the first holder 61A to the first holding bars 71 by using the grooves of the pair of connecting arms 631A. Therefore, a height adjustment can be made by shifting a position of the first holder 61A along the grooves.

Moreover, either of the connecting arms 631A is provided with a state detection unit for detecting whether or not the first holder 61A is connected to the front side of the second holder 62A.

This state detection unit can be realized by providing a micro switch or the like, which comes in contact with the first holding bar 71 at the time of connecting, for example, inside, or in proximity to, the groove of one of the connecting arms 631A, This state detection unit also transmits the detection result to the console C by wireless communication or wired communication,

### Radiographic Image Capturing Operation

Radiographic image capturing operation that uses the above-described radiographic image capturing stain 60A will be described.

The radiographic image capturing devices P, which are stored in the first and second holders 61A, 62A of the radiographic image capturing stand 60A, are provided with respective dongles, each of which stores individual identification information. The identification information of each of four radiographic image capturing devices P is registered, in the console C, by being associated with information indicating where each of the radiographic image capturing devices is stored, that is to say, any one of the first holder 61A, and upper, middle and lower slots of the second holder 62A.

In addition, when single image capturing is performed, the first holder 61A that holds the radiographic image capturing device P is connected to the front side of the second holder 62A.

Further, a connection state of the first holder 61A is detected by the state detection unit, and is then transmitted to the console C.

Consequently, even when the radiographic image capturing device P stored in the second holder 62A is exposed to a radiation ray, with the result that radiographic image data is generated and is then automatically transmitted, the console C is capable of preventing the radiographic image data from, for example, being confused or misidentified.

Subsequently, a subject is subjected to height adjustment and the like, and the subject is then exposed to a radiation ray from the radiation irradiation device 52. As the result, the radiographic image capturing device P detects the radiation ray, generates radiographic image data, and then transmits the radiographic image data to the console C together with identification information.

The console C subjects the radiographic image data obtained from the radiographic image capturing device P to predetermined image processing, and then stores the processed image data in the storage unit Cb with the image data associated with identification data of the radiographic image capturing device P.

In addition, when long-length image capturing (one-shot long-length image capturing) is performed, the first holder 61A is disconnected, and the second holder 62A storing three radiographic image capturing devices P, which are inserted through individual slots, is used.

Further, a non-connection state of the first holder 61A is detected by the state detection unit, and is then transmitted to the console C.

Subsequently, a subject is subjected to position adjustment and the like, and the subject is then exposed to a radiation ray from the radiation irradiation device 52. As the result, each of the three radiographic image capturing devices P detects the radiation ray, generates radiographic image data, and then transmits the radiographic image data to the console C together with identification information.

The console C identifies, from accompanying identification information, how the pieces of received radiographic image data are associated with the three radiographic image capturing devices P, and further how the pieces of received radiographic image data are associated with three slots of the second holder 62A. The console C generates pieces of radiographic image data in the order of arrangement at the time of image capturing, subjects the pieces of radiographic image data to usual image processing, and then stores the pieces of radiographic image data in the storage unit Cb by being associated with identification data of the three radiographic image capturing devices P and the arrangement information thereof.

### Technical Effects of Second Embodiment

The above-described radiographic image capturing stand 60A performs single image capturing by using the first holder 61A connected to the front surface side of the second holder 62A. Therefore, as shown in FIG. 20B, the height of the first holder 61A can be easily adjusted to be low. In addition, a sufficient space can be maintained under the first holder 61A.

Accordingly, image capturing of a child, a subject with small stature, and a subject using a wheelchair can also be preferably handled. In addition, disconnecting the first holder 61A also enables long-length image capturing that uses the second holder 62A.

Moreover, the long-length image capturing is performed by using the second holder 62A alone, which does not cause the second holder 62A to be unstable, thereby enabling excellent long-length image capturing to be stably achieved.

Therefore, in the case of the radiographic image capturing stand 60A as well, it is not necessary to individually prepare a radiographic image capturing stand used exclusively for single image capturing, and a radiographic image capturing stand used exclusively for long-length image capturing; and excellent single image capturing and excellent long-length image capturing can be realized even in the case of a narrow space.

Moreover, the first holder 61A is configured to be attachable/detachable by using the coupler 63A, which enables easy and quick switching between a single image capturing state and a long-length image capturing state. This leads to an improvement in the workability at the time of image capturing.

### Others

It should be noted that although the case where the three radiographic image capturing devices P are stored in the second holder 62A has been presented, the number of radiographic image capturing devices P to be stored may be two, or four or more.

When four or more radiographic image capturing devices P are stored, it is preferable that the radiographic image capturing devices P be arranged in a line along the longitudinal direction of the radiographic image capturing device P.

### Third Embodiment

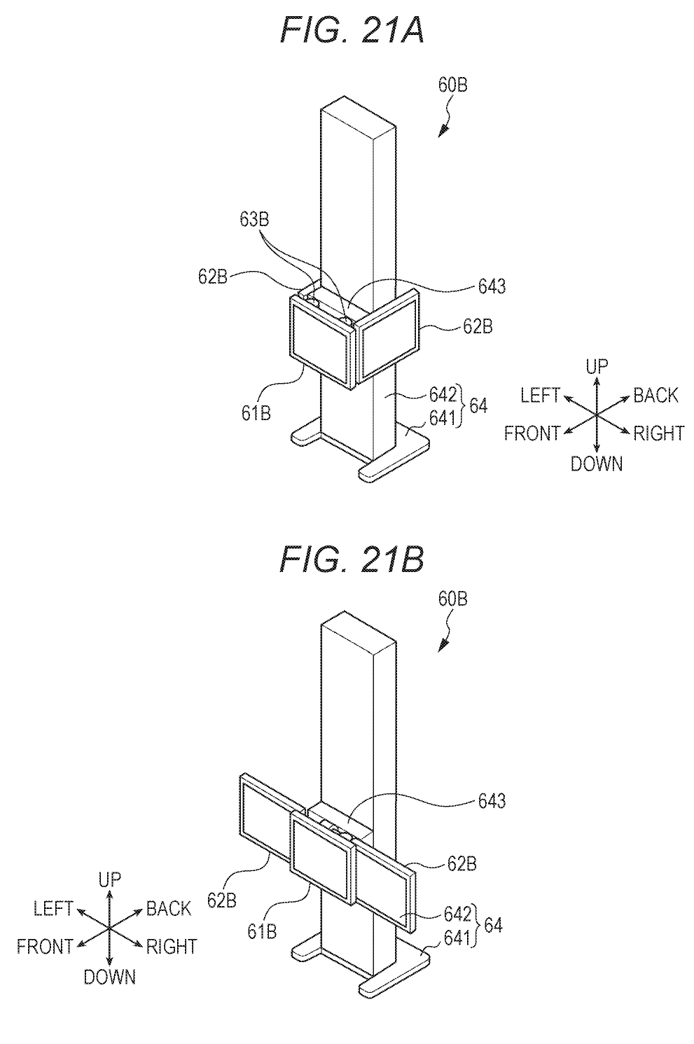

As a third embodiment of the present invention, still another type of radiographic image capturing stand 60B will be described on the basis of FIGS. 21A to 24B. The radiographic image capturing system 50 is equipped with this radiographic image capturing stand 60B as an alternative to the radiographic image capturing stand 60 described above.

It should be noted that with respect to the radiographic image capturing stand 60B, identical reference numerals are used for components identical to those of the radiographic image capturing stand 60, and overlapping explanation will be omitted.

The radiographic image capturing stand 60B includes: a first holder 61B functioning as a first holding part that stores and holds the single radiographic image capturing device P; second holders 62B functioning as two second holding parts that are provided adjacently to the right and left of the first holder 61B respectively, and individually store and hold the single radiographic image capturing device P; couplers 63B that are tunably connected to the second holders 62B respectively so as to switch between a state in which the second holders 62B are arranged in a line with respect to the right and left end parts of the first holder 61B, and face forward, and a state in which the second holders 62B are folded backward; a support stand 64 that supports the first holder 61B; and a holding unit 70 (not illustrated in FIGS. 21A to 24B) for, when a subject is subjected to radiographic image capturing, allowing the subject to hold on thereto.

As with the first holder 61, the first holder 61B is capable of storing and holding the single radiographic image capturing device P from a slot (not illustrated). The feature wherein the first holder 61B is directly supported by the support stand 64 is also the same.

However, as shown in FIGS. 21A and 21B, in a usual holding state, the first holder 61B is supported by the support stand 64 with the longitudinal direction of the first holder 61B (the longitudinal direction of the radiographic image capturing device P stored therein) directed in the right-and-left direction. In addition, in the above-described posture, the first holder 61B may be provided with a chin support part on the upper end part thereof.

The two second holders 62B are both capable of storing and holding the single radiographic image capturing device P from a slot (not illustrated), and are connected to the right and left end parts of the first holder 61B respectively by the couplers 63B each having a hinge structure.

The second holders 62B are tumably connected by the couplers 63B respectively so as to allow the second holders 62B to turn about shafts along the up-and-down direction. As the result, turning the second holders 62B enables a state in which the front faces (radiation incident plane) of the second holders 62B face forward in the same manner as the front face of the first holder 61B (state of FIG. 21B), and a state in which the turning end parts of the second holder 62B are directed backward (state of FIG. 21A) to be switched.

In addition, in a state of facing forward (state of FIG. 21B), the second holders 62B are connected by the couplers 63B respectively in such a manner that the longitudinal directions of the second holders 62B (the longitudinal directions of the radiographic image capturing devices P stored therein) are directed in the right-and-left direction in the same manner as the longitudinal direction of the first holder 61B.

Moreover, in a state in which the second holders 62B face forward, the couplers 63B connect the second holders 62B respectively in such a manner that one end part of each of the radiographic image capturing devices P stored in the second holders 62B respectively and each end part of the radiographic image capturing device P stored in the first holder 61B overlap each other adjacently in the front-and-back direction.

As the result, in the case of long-length image capturing, pieces of radiographic image capturing data obtained by the three radiographic image capturing devices P respectively can be synthesized in a state in which the pieces of data are seamlessly combined.

The support stand 64 is structured in the same manner as that of the radiographic image capturing stand 60. Therefore, the first holder 61B can, be turned about a shaft along the right-and-left direction, and can be turned about a shaft along the front-and-back direction.

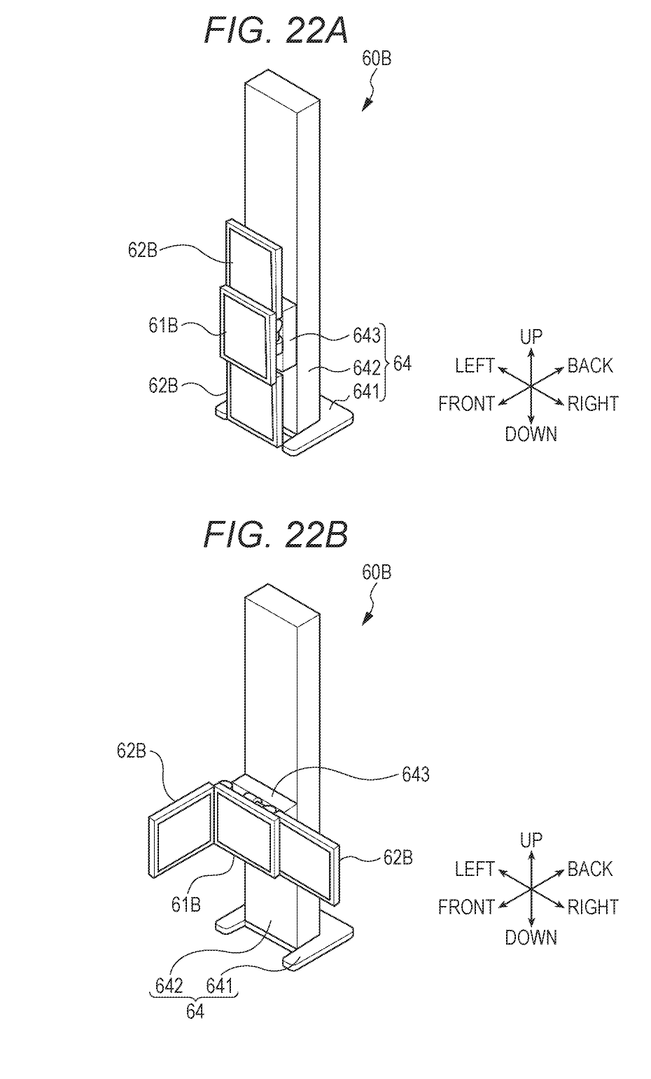

Therefore, as shown in FIG. 22A, the three radiographic image capturing devices P can be vertically arranged in a line by turning the first holder 61B about the shaft along the front-and-back direction by 90° in a state in which the first holder 61B and the two second holders 62B all face forward. Long-length image capturing can be performed in this state.

In addition, the three radiographic image capturing device P arranged in a line can be all directed upward by turning the first holder 61B about the shaft along the right-and-left direction by 90° in a state in which the first holder 61B and the two second holders 62B all face forward. The feature can also be used for radiographic image capturing of a subject who lies on his/her side.

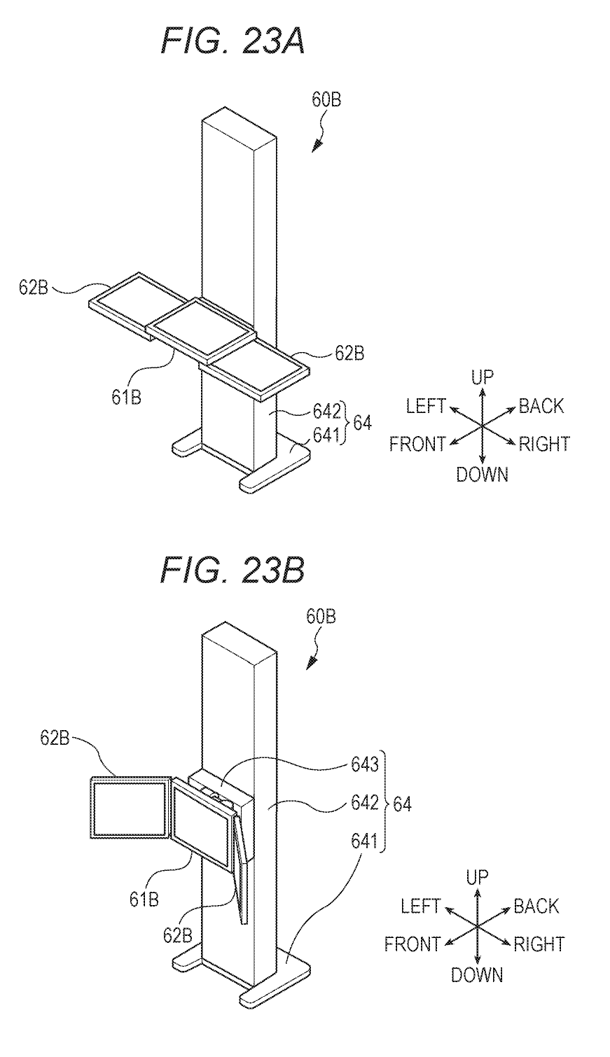

Moreover, as shown in FIGS. 22B and 23B, using turnable support by the support stand 64 and turnable support by each of the couplers 63B in combination also enables radiographic image capturing from different directions to be concurrently performed.

Moreover, the couplers 63B are provided with a state detection unit that detects a change of state between a state in which the second holders 62B and the first holder 61B are directed in the same direction (state of FIG. 22A) and a state in which the respective turning end parts of the second holders 62B are folded backward (state of FIG. 21A).

This state detection unit can be realized by, for example, an encoder that detects a change in angle of a turning shaft in each of the couplers 63B, or a micro switch that switches between ON and OFF by coming in contact with the holder before and after an angle change. This state detection unit also transmits the detection result to the console C by wireless communication or wired communication.

### Radiographic Image Capturing Operation

Radiographic image capturing operation that uses the above-described radiographic image capturing stand 60B will be described.

The radiographic image capturing devices P, which are stored in the first and second holders 61B, 62B of the radiographic image capturing stand 60B, are provided with respective dongles, each of which stores individual identification information.

The identification information of each of three radiographic image capturing devices P is registered, in the console C, by being associated with information indicating where each of the radiographic image capturing devices is stored, that is to say, any one of the first holder 61B, the second holder 62B on the right side, and the second holder 62B on the left side.

In addition, when single image capturing is performed, the first holder 61B that stores and holds the radiographic image capturing device P through a slot is directed forward, and the second holders 62B are then turned in such a manner that the respective turning end parts of the second holders 62B are directed backward (state of FIG. 21A).

Moreover, a direction of the first holder 61B and respective directions of the second holders 62B are detected by the state detection unit, and are then transmitted to the console C.

Subsequently, a subject is subjected to height adjustment and the like, and the subject is then exposed to a radiation ray from the radiation irradiation device 52. As the result, the radiographic image capturing device P detects the radiation ray, generates radiographic image data, and then transmits the radiographic image data to the console C together with identification information.

The console C subjects the radiographic image data obtained from the radiographic image capturing device P to predetermined image processing, and then stores the processed image data in the storage unit Cb with the image data associated with identification data of the radiographic image capturing device P.

In addition, when long-length image capturing (one-shot long-length image capturing) is performed, the second holders 62B that store the radiographic image capturing devices P inserted through slots respectively are turned forward, and the first holder 61B is turned about the shaft in the front-and-back direction by 90° by the support stand 64 to bring the first holder 61B and the two second holders 62B into a state of being vertically arranged side by side (state of FIG. 22A).

Moreover, a direction of the first holder 61B and respective directions of the second holders 62B are detected by the state detection unit, and are then transmitted to the console C.

Subsequently, a subject is subjected to position adjustment and the like, and the subject is then exposed to a radiation ray from the radiation irradiation device 52. As the result, each of the three radiographic image capturing devices P detects the radiation ray, generates radiographic image data, and then transmits the radiographic image data to the console C together with identification information,

The console C identifies, from accompanying identification information, how the pieces of received radiographic image data are associated with the three radiographic image capturing devices P, and further how the pieces of received radiographic image data are associated with the first holder 61B and the right and left second holders 62B. The console C generates pieces of radiographic image data in the order of arrangement at the time of image capturing, subjects the pieces of radiographic image data to usual image processing, and then stores the pieces of radiographic image data in the storage unit Cb by being associated with identification data of the three radiographic image capturing devices P and the arrangement information thereof.

### Technical Effects of Third Embodiment

The above-described radiographic image capturing stand 60B performs single image capturing by using the first holder 61B with the second holders 62B on both the right and left sides turned backward. Therefore, the height of the first holder 61B can be easily adjusted to be low. In addition, a sufficient space can be maintained under the first holder 61B.

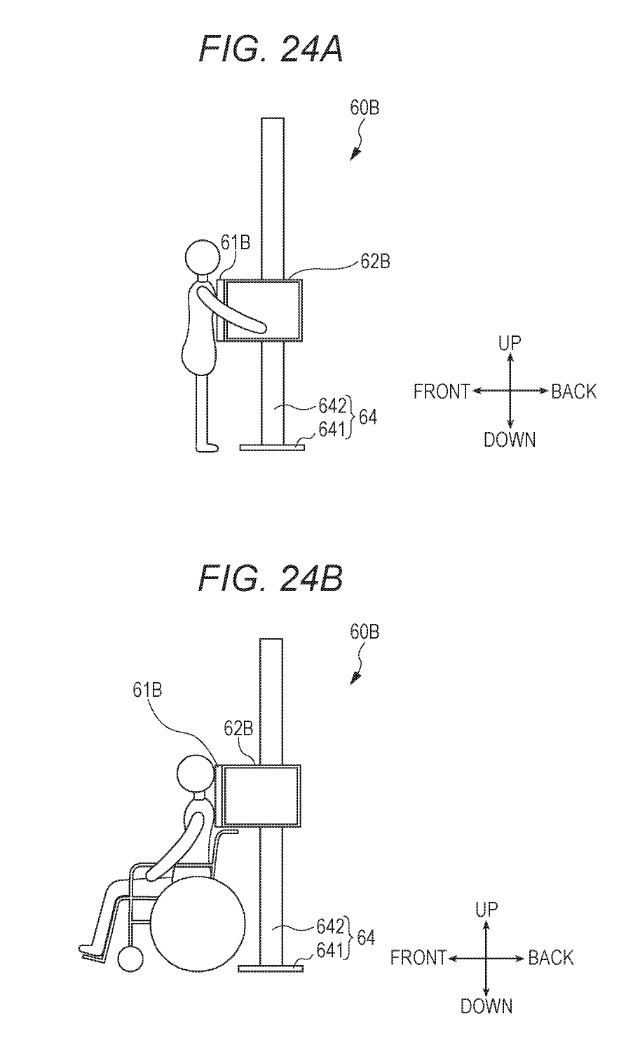

Therefore, as shown in FIGS. 24A and 24B, image capturing of a child, a subject with small stature, and a subject using a wheelchair can also be preferably handled.

In addition, by turning the second holders 62B in such a manner that the second holders 62B each face forward, the first holder 61B and the two second holders 62B are brought into a state of being arranged in a line, and therefore long-length image capturing can also be performed.

Moreover, the second holders 6213 are connected to the first holder 61B by the couplers 63B respectively. Therefore, interposing a friction body or a braking material into a turning part or providing a stopper enables the postures of the second holders 62B to be stably maintained, and consequently excellent long-length image capturing can be stably achieved.

Therefore, in the case of the radiographic image capturing stand 60B as well, it is not necessary to individually prepare a radiographic image capturing stand used exclusively for single image capturing, and a radiographic image capturing stand used exclusively for long-length image capturing; and excellent single image capturing and excellent long-length image capturing can be realized even in the case of a narrow space.

Furthermore, the turning operation of turning the second holders 62B enables easy and quick switching between a single image capturing state and a long-length image capturing state. This leads to an improvement in the workability at the time of image capturing.

### Others

It should be noted that although the second holders 62B are provided adjacently to both sides of the first holder 61B respectively, the second holder 62B may be provided on either of the sides only.

In addition, the configuration of the second holders 62B is not limited to the case where the second holders 62B each store only one radiographic image capturing device P. The second holders 62B may be configured to store two or more radiographic image capturing devices P in a state of being arranged in a line.

### Fourth Embodiment

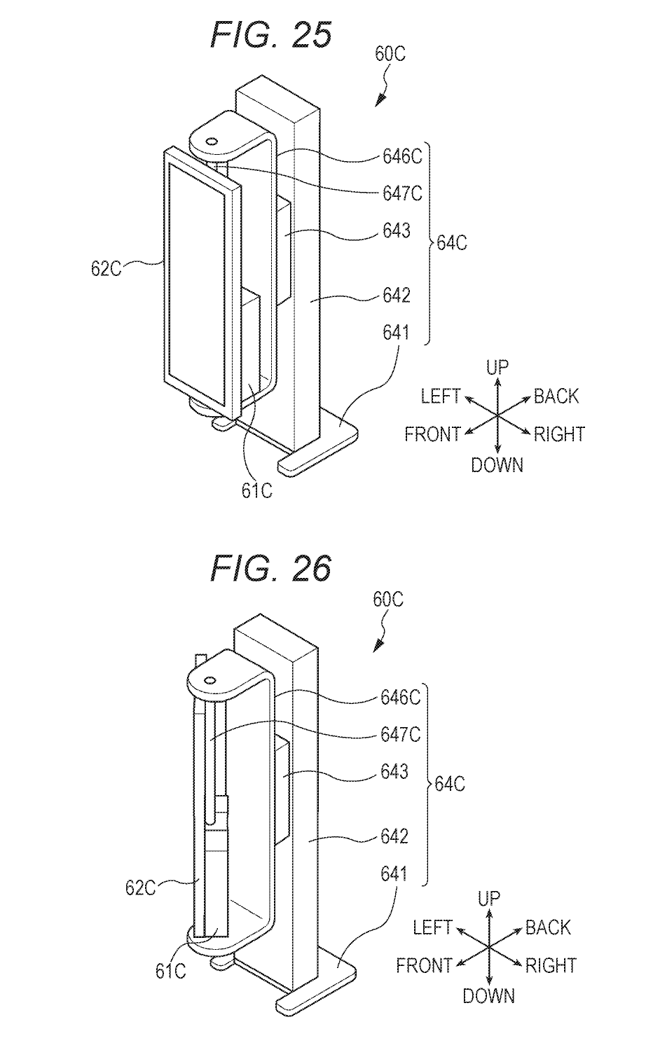

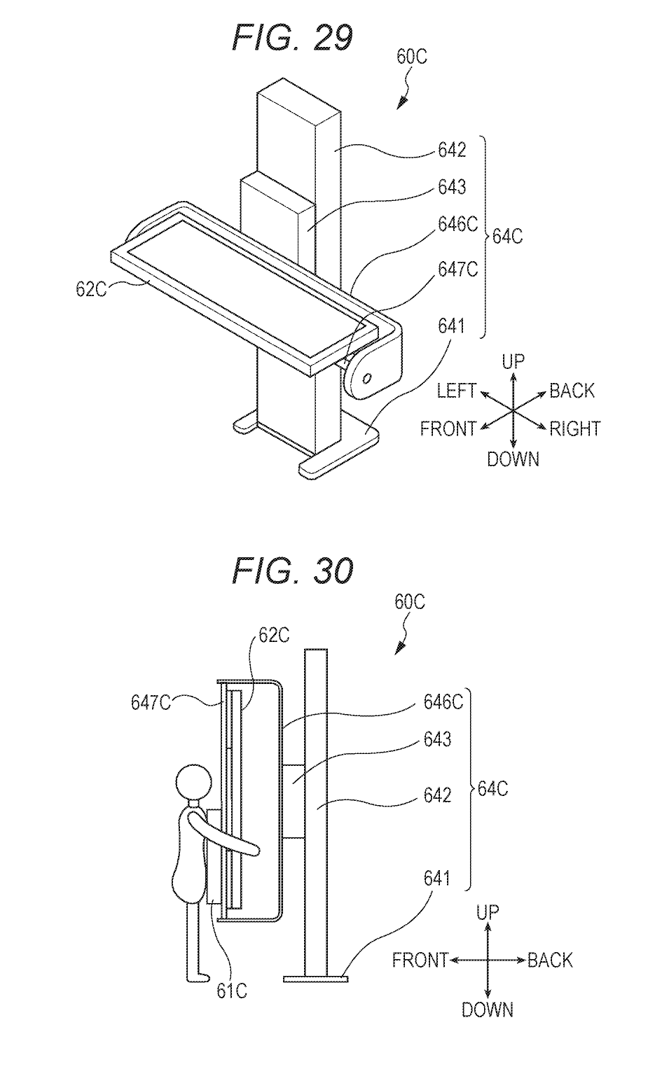

As a fourth embodiment of the present invention, a further type of radiographic image capturing stand 60C will be described on the basis of FIGS. 25 to 31. The radiographic image capturing system 50 is equipped with this radiographic image capturing stand 60C as an alternative to the radiographic image capturing stand 60 described above.

It should be noted that with respect to the radiographic image capturing stand 60C, identical reference numerals are used for components identical to those of the radiographic image capturing stand 60, and overlapping explanation will be omitted.

The radiographic image capturing stand 60C includes: a first holder 61C functioning as a first holding part that stores and holds the single radiographic image capturing device P; a second holder 62C functioning as a second holding part that stores and holds three radiographic image capturing devices P in such a manner that the three radiographic image capturing devices P are vertically arranged in a line; a support stand 64C functioning as a supporter that rotatably supports the first holder 61C and the second holder 62C in such a manner that the first holder 61C and the second holder 62C are arranged back to back with each other so as to direct the radiation incident plane of the radiographic image capturing device P of the first holder 61C and the radiation incident plane of the radiographic image capturing device P of the second holder 62C in directions opposite to each other, and the radiation incident plane of the radiographic image capturing device P of the first holder 61C and the radiation incident plane of the radiographic image capturing device P of the second holder 62C selectively face forward; and a holding unit 70 (not illustrated in FIGS. 25 to 31) for, when a subject is subjected to radiographic image capturing, allowing the subject to hold on thereto.

As with the first holder 61, the first holder 61C is capable of storing and holding the single radiographic image capturing device P from a slot (not illustrated). In addition, the first holder 61C holds the radiographic image capturing device P with the longitudinal direction of the radiographic image capturing device P stored therein aligned along the up-and-down direction.

Moreover, as with the first holder 61, the upper end part of the first holder 61C is provided with a chin support part 612C.

The second holder 62C differs from the above-described second holder 62 in that the three radiographic image capturing devices P are vertically stored side by side, and that the second holder 62C is directly supported by the support stand 64C. Other than the above points of difference, the second holder 62C is the same as the above-described second holder 62. The second holder 62C is provided with three slots (not illustrated), and stores three radiographic image capturing devices P therein. Therefore, in the case of long-length image capturing, the second holder 62C alone is capable of performing image capturing.

in addition, since the second holder 62A stores the three radiographic image capturing devices P, it is not necessary to store the three radiographic image capturing devices P with the upper end part of the radiographic image capturing device P, which is stored in the uppermost part, protruded from the upper end part of the holder toward the outside as with the second holder 62. All of the radiographic image capturing devices P are stored inside the holder.

The support stand 64C differs from the above-described support stand 64 in that as an alternative to the support arm 644, the support stand 64C is provided with: a U-shaped frame body 646C that is turnably supported by the above-described holder lifting and lowering body 643 so as to allow the U-shaped frame body 646C to turn about a shaft along the front-and-back direction, and that is long in the up-and-down direction; and a turning shaft 647C that is provided inside the frame body 646C, and that integrally turns the first holder 61C and the second holder 62C which are arranged back to back with each other.

The U-shaped frame body 646C is longer than the second holder 62C in the up-and-down direction, and rotatably supports the turning shalt 647C inside the U-shaped frame body 646C with the turning shaft 647C directed in the up-and-down direction. The first holder 61C and the second holder 62C are integrally connected to each other in a state in which the front faces (radiation incident planes) of the stored radiographic image capturing devices P are directed in directions opposite to each other. The turning shaft 647C integrally and tumably supports the first holder 61C and the second holder 62C so as to allow the first holder 61C and the second holder 62C to turn about the shaft along the up-and-down direction,

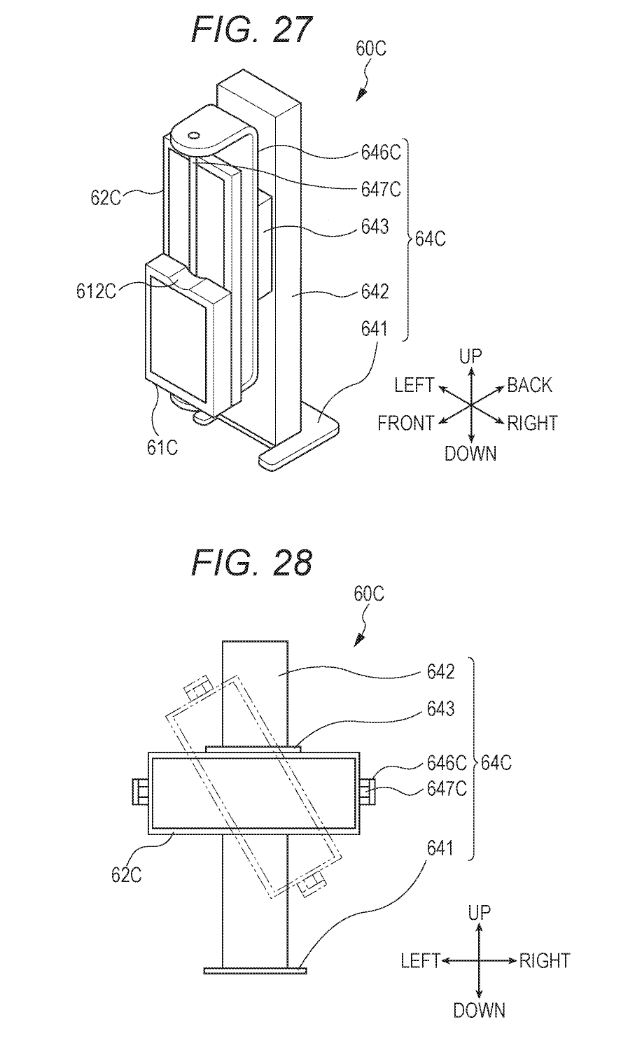

As the result, integrally turning the first holder 61C and the second holder 62C about the turning shaft 647C as shown in FIG. 26 enables selective switching to a state in which either of the first and second holders 61C, 62C faces forward between a state shown in FIG. 25 in which the second holder 62C faces forward and a state shown in FIG. 27 in which the first holder 61C faces forward.

In addition, the support stand 64C is provided with a state detection, unit that detects a change of state between a state in which the first holder 61C faces forward (shown in FIG. 27) and a state in which the second holder 62C faces forward (shown in FIG. 25).

This state detection unit can be realized by, for example, an encoder for detecting a change in angle of the turning shaft 647C.

This state detection unit also transmits the detection result to the console C by wireless communication or wired communication.

Further, as with the above-described support stand 64, the support stand 64C is capable of turning the frame body 646C about a shaft along the front-and-back direction.

Therefore, as shown in FIG. 28, the longitudinal direction of the second holder 62C can be horizontally directed, which enables radiographic image capturing of a subject who lies in a bed by being horizontally irradiated with a radiation ray.

Furthermore, from the state of FIG. 28, the first holder 61C and the second holder 62C are turned about the turning shaft 647C to cause the second holder 62C to face upward. This also enables radiographic image capturing of a subject who lies on his/her side.

### Radiographic Image Capturing Operation

Radiographic image capturing operation that uses the above-described radiographic image capturing stand 60C will be described.

The radiographic image capturing devices P, which are stored in the first and second holders 61C, 62C of the radiographic image capturing stand 60C, are provided with respective dongles, each of which stores individual identification information. The identification information of each of four radiographic image capturing devices P is registered, in the console C, by being associated with information indicating where each of the radiographic image capturing devices is stored, that is to say, any one of the first holder 61C, and the upper, middle and lower slots of the second holder 62C.

In addition, when single image capturing is performed, the first holder 61C and the second holder 62C are turned in such a manner that the first holder 61C that stores and holds the radiographic image capturing device P trough a slot faces forward (state of FIG. 27).

Moreover, a direction of the first holder 61C and a direction of the second holder 62C are detected by the state detection unit, and are then transmitted to the console C.

Subsequently, a subject is subjected to height adjustment and the like, and the subject is then exposed to a radiation ray from the radiation irradiation device 52. As the result, the radiographic image capturing device P detects the radiation ray, generates radiographic image data, and then transmits the radiographic image data to the console C together with identification information.

The console C subjects the radiographic image data obtained from the radiographic image capturing device P to predetermined image processing, and then stores the processed image data in the storage unit Cb with the image data associated with identification data of the radiographic image capturing device P.

In addition, when long-length image capturing (one-shot long-length image capturing) is performed, the first holder 61C and the second holder 62C are turned in such a manner that the second holder 62C storing the radiographic image capturing devices P, which are inserted through individual slots, faces forward (state of FIG. 25). Moreover, a direction of the first holder 61C and a direction of the second holder 62C are detected by the state detection unit, and are then transmitted to the console C.

Subsequently, a subject is subjected to position adjustment and the like, and the subject is then exposed to a radiation ray from the radiation irradiation device 52. As the result, each of the three radiographic image capturing devices P detects the radiation ray, generates radiographic image data, and then transmits the radiographic image data to the console C together with identification information.

The console C identifies, from accompanying identification information, how the pieces of received radiographic image data are associated with the three radiographic image capturing devices P, and further how the pieces of received radiographic image data are associated with the upper, middle and lower slots of the second holder 62C, through which the three radiographic image capturing devices P are inserted to be stored. The console C generates pieces of radiographic image data in the order of arrangement at the time of image capturing, subjects the pieces of radiographic image data to usual image processing, and then stores the pieces of radiographic image data in the storage unit Cb by being associated with identification data of the three radiographic image capturing devices P and the arrangement information thereof.

### Technical Effects of Fourth Embodiment

The above-described radiographic image capturing stand 60C performs single image capturing by using the first holder 61C with the first holder 61C facing forward. Therefore, the height of the first holder 61C can be easily adjusted to be low. In addition, a sufficient space can be maintained under the first holder 61C.

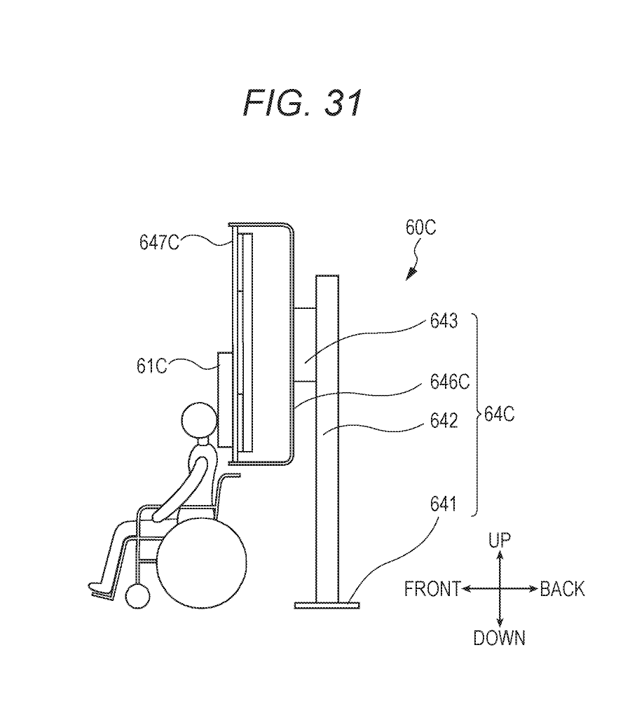

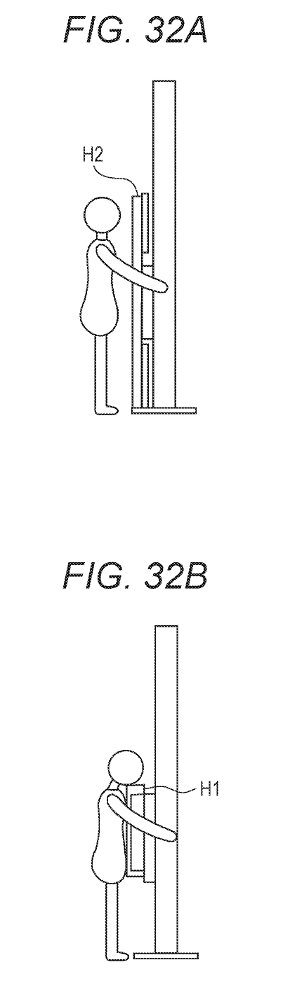

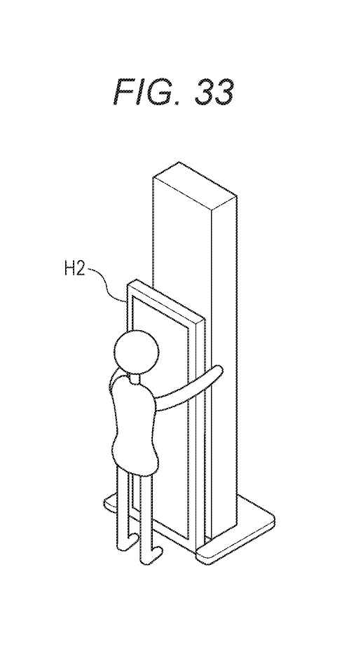

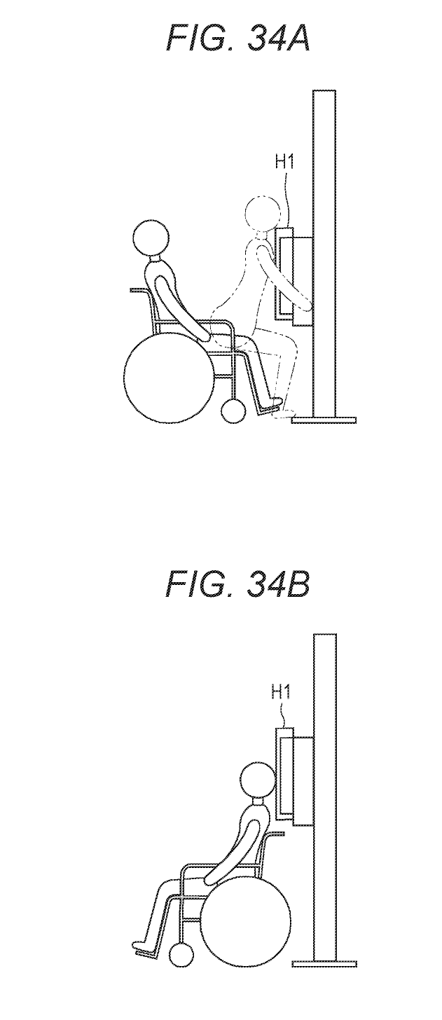

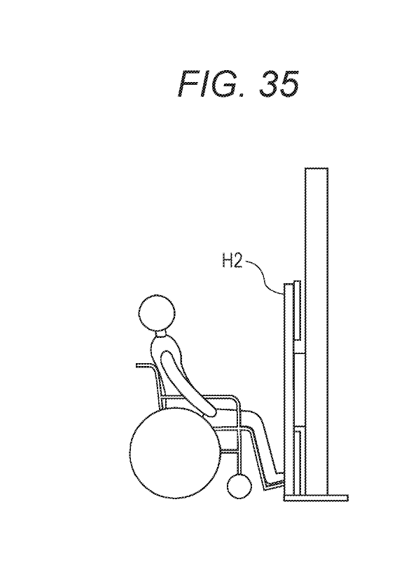

Therefore, as shown in FIGS. 30 and 31, image capturing of a child, a subject with small stature, and a subject using a wheelchair can also be preferably handled.

In addition, by turning the first holder 61C and the second holder 62C so as to cause the second holder 62C to face forward, long-length image capturing can also be performed.

Moreover, the first holder 61C and the second holder 62C are tunably supported by the support stand 64C so as to allow the first holder 61C and the second holder 62C to turn about the shalt along the up-and-down direction. Therefore, by providing a friction body and a braking material around the turning shalt 647C, or by providing a stopper, the directions of the first holder 61C and the second holder 62C can be stably maintained, thereby enabling excellent long-length image capturing to be stably achieved.

Therefore, in the case of the radiographic image capturing stand 60C as well, it is not necessary to individually prepare a radiographic image capturing stand used exclusively for single image capturing, and a radiographic image capturing stand used exclusively for long-length image capturing; and excellent single image capturing and excellent long-length image capturing can be realized even in the case of a narrow space.

Furthermore, the turning operation of turning the first holder 61C and the second holder 62C enables easy and quick switching between a single image capturing state and a long-length image capturing state. This leads to an improvement in the workability at the time of image capturing.

### Others

It should be noted that although the case where the three radiographic image capturing devices P are stored in the second holder 62C has been presented, the number of radiographic image capturing devices P to be stored may be two, or four or more, When four or more radiographic image capturing devices P are stored, it is preferable that the radiographic image capturing devices P be arranged in a line along the longitudinal direction of the radiographic image capturing device P.

In addition, in the above-described embodiments, the radiographic image capturing stands 60, 60A, 60B, 60C are all presented in a mode in which the radiographic image capturing device P provided with the housing 2, which functions as a radiation detection unit, can be freely stored in, and removed from, the first holder and the second holder, The present invention is not limited to this mode.

For example, the first holder and the second holder may be each configured to have built-in only internal components inside the housing 2 of the radiographic image capturing device P as a radiation detection unit, and thus not allowing the internal components to be freely stored and removed.

In other words, the first holder and the second holder may be each configured to substantially become the radiographic image capturing device itself.

Moreover, in the above-described embodiments, the radiographic image capturing stands 60, 60A, 60B, 60C are all presented as a case where the radiographic image capturing device P has a rectangular shape having long sides, and is stored in the first and second holders with the longitudinal direction of the radiographic image capturing device P aligned in the up-and-down direction. However, the present invention is not limited to this case. The radiographic image capturing device P may be stored in the first and second holders with the lateral direction of the radiographic image capturing device P aligned in the up-and-down direction.

Furthermore, the radiographic image capturing device P may be configured to have a square shape with four equal sides, In this case, when the radiographic image capturing device P has a directional dependence in the up-and-down direction, the radiographic image capturing device P is aligned thereto. When the radiographic image capturing device P has no directional dependence, the radiographic image capturing device P may be stored in the first and second holders in any direction without the alignment based on the directional dependence.

Although embodiments of the present invention have been described and illustrated in detail, it is clearly understood that the same is by way of illustration and example only and not limitation, the scope of the present invention should be interpreted by terms of the appended claims.

What is claimed is:

1. A radiographic image capturing stand comprising:
a plurality of radiographic image capturing parts, each of which forms a radiographic image by being exposed to a radiation ray, and the radiographic image capturing stand being capable of switching between a single image capturing state in which image capturing is performed with the single radiographic image capturing part held with respect to one exposure to a radiation ray, and a long-length image capturing state in which image capturing is performed with the plurality of radiographic image capturing parts held side by side with respect to one exposure to a radiation ray.

2. The radiographic image capturing stand according to claim 1, further comprising:
a first holding part that stores and holds the single radiographic image capturing part;
a second holding part that stores and holds the plurality of radiographic image capturing parts side by side; and
a coupler that removably fixes and mounts the second holding part to an outer edge part of the first holding part.

3. The radiographic image capturing stand according to claim 1, further comprising:
a first holding part that stores and holds the single radiographic image capturing part;
a second holding part that stores and holds the plurality of radiographic image capturing parts side by side; and
a coupler that removably fixes and mounts the first holding part to the front side of the second holding part.

4. The radiographic image capturing stand according to claim 1, further comprising:
a first holding part that stores and holds the single radiographic image capturing part;
a second holding part that is provided adjacently to the first holding part, and that stores and holds the other radiographic image capturing parts; and
a coupler that tunably connects the second holding part to one end part of the first holding part so as to allow the second holding part to turn about the one end part of the first holding part, thereby enabling a state in which the second holding part and the first holding part both face forward and are arranged to form a line, and a state in which the second holding part is folded back to be switched.

5. The radiographic image capturing stand according to claim 1, further comprising:
a first holding part that stores and holds the single radiographic image capturing part;
a second holding part that stores and holds the plurality of radiographic image capturing parts side by side; and
a supporter that rotatably supports the first holding part and the second holding part in such a manner that the first holding part and the second holding part are arranged back to back with each other so as to direct the radiographic image capturing part of the first holding part and the radiographic image capturing part of the second holding part in directions opposite to each other, and the radiographic image capturing part of the first holding part and the radiographic image capturing part of the second holding part selectively face forward.

6. The radiographic image capturing stand according to claim 1, further comprising:
a grip that allows a subject who is subjected to radiographic image capturing to hold on thereto.
